(19) United States
(12) Patent Application Publication
(10) Pub. No.: US 2019/0193079 A1
(43) Pub. Date: Jun. 27, 2019

(54) SYSTEMS AND METHODS FOR HEATING BIOLOGICAL SAMPLES

(71) Applicant: Coyote Bioscience USA Inc., Campbell, CA (US)

(72) Inventors: Jesus Ching, Saratoga, CA (US); Phillip You Fai Lee, South San Francisco, CA (US); Chen Li, Los Gatos, CA (US)

(21) Appl. No.: 16/137,351

(22) Filed: Sep. 20, 2018

Related U.S. Application Data
(63) Continuation of application No. PCT/US2017/025393, filed on Mar. 31, 2017.
(60) Provisional application No. 62/318,077, filed on Apr. 4, 2016.
(60) Provisional application No. 62/419,079, filed on Nov. 8, 2016.

(51) Int. Cl. B01L 7/00; C12Q 1/686; B01L 3/00

(52) U.S. Cl. CPC B01L 7/52; C12Q 1/686; G01N 1/30; B01L 7/54; B01L 3/50851

(57) ABSTRACT

The present disclosure provides systems and methods for heating biological samples. Methods and systems of the present disclosure may be used for performing nucleic acid amplification to identify one or more targets in a nucleic acid sample of a subject.

### CROSS-REFERENCE

This application is a continuation of Patent Cooperation Treaty Application No. PCT/US2017/025393, filed on Mar. 31, 2017, which claims priority to U.S. Provisional Patent Application No. 62/318,077, filed Apr. 4, 2016, and U.S. Provisional Patent Application No. 62/419,079, filed Nov. 8, 2016, each of which is entirely incorporated herein by reference.

### BACKGROUND

The addition of heat to a biological sample may be a required component for processing the biological sample, such as in preparing molecules in the biological sample for downstream analysis or conducting a chemical or biological reaction on the biological sample. Chemical and biological reactions conducted on a biological sample can be useful tools in diagnostics. However, the utility of many diagnostic tests and procedures demands relatively quick sample-to-answer processing times. Slow heating times, slow temperature ramp rates or associated losses of heat to the surrounding environment may render many heating systems and methods currently available incompatible with some diagnostics.

### SUMMARY

Recognized herein is a need for faster, more reliable, more efficient heating systems and methods for processing a biological sample.

The present disclosure provides systems and methods for processing a biological sample. Systems and methods of the present disclosure may include or make use of inductive heating for heating the biological sample.

An aspect of the disclosure provides a method for processing a biological sample. The method comprises (a) providing a solution comprising the biological sample and one or more heating elements in a vessel. The one or more heating elements are dispersed in the solution the one or more heating elements generate heat upon inductive coupling to a magnetic field. The method also comprises (b) bringing the solution in contact with the magnetic field; and (c) subjecting the solution comprising the biological sample to heating upon inductive coupling of the one or more heating elements to the magnetic field.

In some embodiments, at least a subset of the heating elements is floating or suspended in the solution. In some embodiments, at least a subset of the one or more heating elements is dissolved in the solution. In some embodiments, the vessel is selected from the group consisting of a tube, a cuvette, a chamber, a beaker, a reservoir, a flow channel, a capillary tube, a well, multi-well plate, a bottle and a flask. In some embodiments, the solution comprises one or more droplets. In some embodiments, the one or more droplets comprise aqueous droplets in an emulsion. In some embodiments, at least a subset of the one or more heating elements is in a given one of the one or more droplets.

In some embodiments, the one or more heating elements generate heat upon flow of eddy current through each of the one or more heating elements. In some embodiments, the one or more heating elements comprise particles, such as, for example, nanoparticles. In some embodiments, the one or more heating elements comprise one or more materials selected from the group consisting of carbon, iron, copper, aluminum, chromium and nickel. In some embodiments, each of the one or more heating elements comprises a polymer and at least one magnetically-active material supported by the polymer. The least one magnetically-active material generates heat upon inductive coupling to the magnetic field. In some embodiments, the one or more heating elements comprise a material comprising at least one free electron.

In some embodiments, the method further comprises providing the magnetic field through a permanent magnet. In some embodiments, the method further comprises providing the magnetic field through an electromagnet. In some embodiments, the electromagnet comprises one or more coils. In some embodiments, at least a portion of the electromagnet is shielded.

In some embodiments, (a) and (b) are performed simultaneously. In some embodiments, the method further comprises (d) subjecting the solution to cooling. In some embodiments, the method further comprises subjecting the solution to cooling upon decoupling of the one or more heating elements from the magnetic field. In some embodiments, the method further comprises subjecting the solution to cooling by positioning the solution in a cooling zone comprising a cooling unit. In some embodiments, the cooling occurs via convection. In some embodiments, the method further comprises repeating (b)-(d), thereby thermal cycling the solution. In some embodiments, the method further comprises conducting a nucleic acid amplification reaction(s) in the solution with the aid of the thermal cycling. The nucleic acid amplification reaction(s) can comprise polymerase chain reaction (PCR) or a variant thereof.

In some embodiments, the solution comprises components necessary for conducting a chemical or biological reaction on the biological sample. In some embodiments, the components comprise a primer and polymerizing enzyme. In some embodiments, the method further comprises conducting the chemical or biological reaction. In some embodiments, the method further comprises detecting a signal indicative of the chemical or biological reaction. In some embodiments, the biological sample comprises a nucleic acid molecule.

The target nucleic acid molecule may be associated with a disease. In some embodiments, the disease is associated with a virus. The virus may be an RNA virus or a DNA virus. For example, the virus may be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, Coxsackie virus, and Varicella virus. The influenza virus may be selected from the group consisting of H1N1 virus, H3N2 virus, H7N9 virus and H5N1 virus. The adenovirus may be adenovirus type 55 (ADV55) or adenovirus type 7 (ADV7). The hepatitis C virus may be armored RNA-hepatitis C virus (RNA-HCV). The Coxsackie virus may be Coxsackie virus A16.

In some embodiments, the disease is associated with a pathogenic bacterium or a pathogenic protozoan. The pathogenic bacterium may be a gram-positive or gram-negative pathogenic bacterium. The pathogenic bacterium may be selected from the group consisting of Staphylococcus aureus, Listeria monocytogenes, Escherichia coli, Enterobacter sakazakii, Vibrio Parahemolyticus, and Shigella spp. In some embodiments, the pathogenic bacterium is Mycobacterium tuberculosis. In some embodiments, the pathogenic bacterium is Salmonella. The pathogenic protozoan may be Plasmodium.

In some embodiments, in (c), a temperature of the at least a subset of the plurality of partitions is raised at a rate of at least about 50° C. per second. In some embodiments, a volume of the solution is at most about 10 milliliters (mL).

An additional aspect of the disclosure provides a system for processing a biological sample. The system comprises a vessel that holds a solution comprising the biological sample and one or more heating elements. The one or more heating elements generate heat upon inductive coupling to a magnetic field. The system also includes a magnetic field application unit that provides a magnetic field to the solution; and a controller operatively coupled to the magnetic field application unit. The controller is programmed to direct the magnetic field application unit to apply the magnetic field to the solution, thereby subjecting the solution to heating upon inductive coupling of the one or more heating elements to the magnetic field.

In some embodiments, at least a subset of the one or more heating elements is floating or suspended in the solution. In some embodiments, at least a subset of the one or more heating elements is dissolved in the solution. In some embodiments, the vessel is selected from the group consisting of a tube, a cuvette, a chamber, a beaker, a reservoir, a flow channel, a capillary tube, a well, a multi-well plate, a bottle and a flask. In some embodiments, the vessel has a volumetric capacity of at most about 10 milliliter (mL).

In some embodiments, the solution comprises one or more droplets. In some embodiments, at least a subset of the one or more heating elements is in a given one of the one or more droplets. In some embodiments, the one or more heating elements generate heat upon flow of eddy current through each of the one or more heating elements. In some embodiments, the one or more heating elements comprise particles such as, for example, nanoparticles. In some embodiments, the one or more heating elements comprise one or more materials selected from the group consisting of carbon, iron, copper, aluminum, chromium and nickel. In some embodiments, each of the one or more heating elements comprises a polymer and at least one magnetically-active material supported by the polymer. The at least one magnetically-active material generates heat upon inductive coupling to the magnetic field. In some embodiments, the one or more heating elements comprise a material comprising at least one free electron.

In some embodiments, the magnetic field application unit comprises a permanent magnet. In some embodiments, the magnetic field application unit comprises an electromagnet. In some embodiments, the electromagnet comprises one or more coils. In some embodiments, at least a portion of the electromagnet is shielded. In some embodiments, the electromagnet is in electrical communication with circuitry that provides electrical current to the electromagnet. In some embodiments, the system further comprises a cooling unit that subjects the solution to cooling. In some embodiments, the system further comprises a cooling unit that provides cooling to a cooling zone. In some embodiments, the cooling unit is selected from the group consisting of a wind tunnel, a convective cooling unit, a cooling block, an evaporative cooling unit, a thermoelectric device (e.g., a Peltier element, indium tin oxide, etc.) and a cooling bath unit.

In some embodiments, the controller comprises one or more computer processors that are individually or collectively programmed to alternately and sequentially position the solution in the magnetic field and a cooling zone. In some embodiments, the vessel is rotatable with respect to the magnetic field application unit, or vice versa. In some embodiments, the solution comprises components necessary for conducting a chemical or biological reaction on the biological sample. In some embodiments, the chemical or biological reaction comprises a nucleic acid amplification reaction. In some embodiments, the components comprise a primer and a polymerizing enzyme. In some embodiments, the nucleic acid amplification reaction comprises polymerase chain reaction (PCR) or a variant thereof. In some embodiments, the biological sample comprises a nucleic acid molecule. In some embodiments, the system further comprises a detector. During detection, the detector detects a signal from the solution that is indicative of a chemical or biological reaction on the biological sample. In some embodiments, the detector may be integral with the vessel holding a solution. In some embodiments the detector may be angularly separated from the vessel. In some embodiments the detector may be operatively coupled with the vessel. In some embodiments the detector is operatively coupled to at least a first thermal zone such that as a detectable sample is brought into at least a first thermal zone, the detectable sample is detected by the detector. In some embodiments the controller positions the solution in sensing communication with the detector. The solution and the detector may be brought into sensing communication via translation for the solution with respect to the detector (and/or vice versa) or via a rotation of the solution with respect to the detector (and/or vice versa), or any combination thereof. The axes of translation and the axes of rotation may be with respect to any characteristic axis of the detector (e.g., with respect to the axis defined by the optical communication path, with respect to the axis perpendicular to the optical communication path, etc.).

An additional aspect of the disclosure provides a method for processing a biological sample. The method comprises: (a) providing a plurality of partitions, where at least a subset of the plurality of partitions comprises the biological sample or a portion thereof; and (b) subjecting the at least a subset of the plurality of partitions to inductive heating at a frequency from about 1 Hz to 10,000 Hz.

In some embodiments, the plurality of partitions is contained in a volume of at most about 10 milliliters. In some embodiments, a volume of a given one of the plurality of partitions is at most about 20 microliters. In some embodiments, the plurality of partitions comprises at least about 1,000 partitions. In some embodiments, the plurality of partitions is contained in a vessel. In some embodiments, the vessel has a volumetric capacity of at most about 10 milliliters. In some embodiments, the vessel is selected from the group consisting of a tube, a cuvette, a chamber, a beaker, a reservoir, a flow channel, a capillary tube, a well, a multi-well plate, a bottle, and a flask. In some embodiments, the plurality of partitions comprises wells. In some embodiments, the plurality of partitions comprises droplets in an emulsion. In some embodiments, (a) further comprises bringing an aqueous phase in contact with a continuous phase to generate the droplets.

In some embodiments, the continuous phase comprises an oil. In some embodiments, the plurality of partitions is contained in a solution in a vessel and the solution and/or a surface of the vessel comprises one or more heating elements that generate heat upon inductive coupling to a magnetic field. In some embodiments, at least a subset of the one or more heating elements is dissolved or suspended in the solution. In some embodiments, at least a subset of the one or more heating elements is in a given one of the plurality of partitions. In some embodiments, the one or more heating elements comprise particles. In some embodiments, the one or more heating elements comprise one or more materials selected from the group consisting of carbon, iron, copper, aluminum, chromium and nickel. In some embodiments, each of the one or more heating elements comprises a polymer and at least one magnetically-active material supported by the polymer. The at least one magnetically-active material generates heat upon inductive coupling to a magnetic field.

In some embodiments, the inductive heating is achieved with the aid of a magnetic field. In some embodiments, the magnetic field is an alternating magnetic field. In some embodiments, the magnetic field is provided by an electromagnet. In some embodiments, the electromagnet comprises one or more coils.

In some embodiments, the method further comprises (c) subjecting the at least a subset of the plurality of partitions to cooling. In some embodiments, the method further comprises subjecting the at least a subset of the plurality of partitions to cooling by positioning the at least a subset of the plurality of partitions in a cooling zone. In some embodiments, the cooling occurs via convection. In some embodiments, the method further comprises repeating (b) and (c), thereby thermal cycling the at least a subset of the plurality of partitions. In some embodiments, the method further comprises conducting a nucleic acid amplification reaction(s) in the at least a subset of the plurality of partitions with the aid of the thermal cycling. In some embodiments, the nucleic acid amplification reaction(s) comprises polymerase chain reaction (PCR) or a variant thereof.

In some embodiments, the at least a subset of the plurality of partitions comprises components necessary for conducting a chemical or biological reaction on the biological sample. In some embodiments, the components comprise a primer and polymerizing enzyme. In some embodiments, the method further comprises conducting the chemical or biological reaction. In some embodiments, the method further comprises detecting a signal indicative of the chemical or biological reaction. In some embodiments, the biological sample comprises a nucleic acid molecule. In some embodiments, in (b), a temperature of the at least a subset of the plurality of partitions is raised at a rate of at least about 50° C. per second.

An additional aspect of the disclosure provides a system for processing a biological sample. The system comprises a plurality of partitions, where at least a subset of the plurality of partitions comprises the biological sample or a portion thereof; an inductive heating unit, where the inductive heating unit inductively heats the at least a subset of the plurality of partitions; and a controller operatively coupled to the inductive heating unit, where the controller is programmed to direct the inductive heating unit to inductively heat the at least a subset of the plurality of partitions at a frequency from about 1 Hz to 10,000 Hz.

In some embodiments, the plurality of partitions is contained in a volume of at most about 10 milliliters. In some embodiments, a volume of a given one of the plurality of partitions is at most about 20 microliters. In some embodiments, the plurality of partitions comprises at least about 1,000 partitions. In some embodiments, the system further comprises a vessel that comprises the plurality of partitions. In some embodiments, the vessel is selected from the group consisting of a tube, a cuvette, a chamber, a beaker, a reservoir, a flow channel, a capillary tube, a well, a multi-well plate, a bottle, and a flask. In some embodiments, the vessel has a volumetric capacity of at most about 10 milliliters. In some embodiments, the vessel is rotatable with respect to the inductive heating unit, or vice versa. In some embodiments, the plurality of partitions comprises wells. In some embodiments, the plurality of partitions comprises droplets in an emulsion.

In some embodiments, the system further comprises (i) a first source of an aqueous phase; (ii) a second source of a continuous phase; and (iii) an emulsion generation unit in fluid communication with the first source and the second source. The emulsion generation unit brings the aqueous phase in contact with the continuous phase to generate the emulsion. In some embodiments, the emulsion generation unit comprises an intersection of a first channel that is in fluid communication with the first source and a second channel that is in fluid communication with the second source. In some embodiments, the continuous phase comprises an oil. In some embodiments, the inductive heating unit generates a magnetic field. In some embodiments, the magnetic field is an alternating magnetic field. In some embodiments, the inductive heating unit comprises an electromagnet. In some embodiments, the electromagnet comprises one or more coils. In some embodiments, at least a portion of the inductive heating unit is shielded. In some embodiments, the inductive heating unit is in electrical communication with circuitry that provides electrical current to the inductive heating unit.

In some embodiments, the system further comprises a vessel comprising the at least a subset of the plurality of partitions and one or more heating elements that generate heat upon inductive coupling to the magnetic field. In some embodiments, at least a subset of the one or more heating elements is dissolved or suspended in a solution. In some embodiments, at least a subset of the one or more heating elements is in a given one of the plurality of partitions. In some embodiments, the one or more heating elements comprise particles. In some embodiments, the one or more heating elements comprise one or more materials selected from the group consisting of carbon, iron, copper, aluminum, chromium and nickel. In some embodiments, each of the one or more heating elements comprises a polymer and at least one magnetically-active material supported by the polymer. The at least one magnetically-active material generates heat upon inductive coupling to the magnetic field.

In some embodiments, a surface of the vessel comprises at least a subset of the one or more heating elements. In some embodiments, the system further comprises a cooling zone that subjects the at least a subset of the plurality of partitions to cooling. In some embodiments, the system further comprises a cooling unit that provides cooling to the cooling zone. In some embodiments, the cooling unit is selected from the group consisting of a wind tunnel, a convective cooling unit, a cooling block, an evaporative cooling unit, a Peltier device, a cooling bath unit. In some embodiments, the controller comprises one or more computer processors that are individually or collectively programmed to alternately and sequentially position the at least a subset of the plurality of partitions in: (i) a heating zone in thermal communication with the inductive heating unit that heats the at least a subset of the plurality of partitions with heat generated by the inductive heating unit; and (ii) the cooling zone.

In some embodiments, the at least a subset of the plurality of partitions comprises components necessary for conducting a chemical or biological reaction on the biological sample. In some embodiments, the chemical or biological reaction comprises a nucleic acid amplification reaction. In some embodiments, the components comprise a primer and polymerizing enzyme. In some embodiments, the nucleic acid amplification reaction comprises polymerase chain reaction (PCR) or a variant thereof. In some embodiments, the biological sample comprises a nucleic acid molecule. In some embodiments, the system further comprises a detector. The detector detects a signal from the at least a subset of the plurality of partitions that is indicative of a chemical or biological reaction on the biological sample. In some embodiments, the detector may be integral with the vessel holding a solution. In some embodiments the detector may be angularly separated from the vessel. In some embodiments the detector may be operatively coupled with the vessel. In some embodiments the detector is operatively coupled to at least a first thermal zone such that as a detectable sample is brought into at least a first thermal zone, the detectable sample is detected by the detector. In some embodiments the controller positions the solution in sensing communication with the detector. The solution and the detector may be brought into sensing communication via translation for the solution with respect to the detector (and/or vice versa) or via a rotation of the solution with respect to the detector (and/or vice versa), or any combination thereof. The axes of translation and the axes of rotation may be with respect to any characteristic axis of the detector (e.g., with respect to the axis defined by the optical communication path, with respect to the axis perpendicular to the optical communication path, etc.).

An additional aspect of the disclosure provides a method for processing a biological sample comprising providing a solution comprising the biological sample in a vessel (the vessel comprising one or more heating elements in thermal communication with the solution) and applying an electric current to one or more heating elements to generate heat. In some embodiments, thermal contact between one or more heating elements and the solution is facilitated by a first fluid. In some embodiments, thermal contact between one or more heating elements and the solution may be facilitated by a second fluid. In some embodiments, the first fluid facilitates thermal contact between at least a first heating element from one or more heating elements and the solution. Furthermore, the second fluid, in some embodiments, facilitates thermal contact between at least a second heating element from the one or more heating elements the solution.

An additional aspect of the disclosure provides a method for processing a biological sample comprising: (a) providing a sample processing unit comprising a plurality of wells and a fluid flow path in fluid communication with the plurality of wells; (b) directing a flow of a plurality of droplets through the fluid flow path to the plurality of wells such that the plurality of droplets is deposited within the plurality of wells; and (c) using at least one heating element to convert electrical energy or electromagnetic energy into thermal energy, thus subjecting the plurality of droplets to heating, and thereby processing the biological sample. In some embodiments, the plurality of wells comprises a sidewall having a material mesh. The material mesh of some embodiments comprises at least one metal such as nickel, chromium, or stainless steel In some embodiments, the plurality of wells is configured to retain the plurality of droplets (the droplets may individually or collectively comprise the biological sample or a portion thereof) during heating of the plurality of droplets. In some embodiments, during heating of the plurality of droplets, at least one heating element is in thermal communication with the plurality of wells. In some embodiments, at least one heating element converts electrical or electromagnetic energy into thermal energy.

In some embodiments, operation (b) of the method may comprises (i) directing the plurality of droplets along the first channel or the second channel or both and (ii) providing a first liquid phase in the first channel and a second liquid phase in the second channel to retain the plurality of droplets in the plurality of wells. The first liquid phase of some embodiments is different from the second liquid phase. In some embodiments, the first liquid phase or the second liquid phase or both is immiscible with the droplet and/or plurality of droplets. In some embodiments, each of the plurality of wells has a first opening in fluid communication with a first channel and a second opening in fluid communication with a second channel. The first opening or the second opening or both may be dimensioned to accept a given droplet from the plurality of droplets.

In some embodiments, operation (c) of the method may comprise each of the plurality of wells containing a single droplet of the plurality of droplets. Furthermore, the plurality of droplets may be subjected to cool, for example by using the sidewall to subject a given droplet from the plurality of droplets to cool. Cooling and/or heating, in some embodiments, may be accomplished through a cooling and/or heating element that is part of a cover adjacent to the plurality of wells either fixedly or temporarily while cooling and/or heating is performed. The cover of some embodiments is optically transparent or semi-transparent (e.g., comprising a material such as indium tin oxide). Moreover, at least one heating element of some embodiments comprises an optically transparent or semi-transparent material (such as indium tin oxide, for example). In some embodiments, the method further comprises providing the cover adjacent to the plurality of wells prior to operation (c).

In some embodiments, the plurality of droplets comprises aqueous droplets in an emulsion. In some embodiments the sample processing unit is part of a chip.

An additional aspect of the disclosure provides a method for performing a nucleic acid amplification reaction on a biological sample comprising: (a) providing a sample processing unit comprising a plurality of wells; (b) dispensing a plurality of droplets into the plurality of wells; and (c) using at least one heating element to convert electrical energy or electromagnetic energy into thermal energy, subjecting the plurality of droplets to heating under conditions sufficient to conduct at least one nucleic acid amplification reaction on the biological sample or portion thereof in the presence of reagents necessary for the nucleic acid amplification reaction, and thereby generating an amplification product(s) of said biological sample or portion thereof.

In some embodiments, each of the plurality of wells is configured to receive and/or confine at most a single droplet of a plurality of droplets. The droplets of some embodiments comprise the biological sample or a portion thereof and reagents necessary for a nucleic acid amplification reaction. In some embodiments, plurality of wells is in thermal communication with at least one heating element. At least one heating element of some embodiments converts electrical or electromagnetic energy into thermal energy. The plurality of wells of may comprise a hygroscopic material to retain the plurality of at least one droplet of the plurality of droplets during the nucleic acid amplification reaction. The plurality of wells may have a first opening in fluid communication with a first channel and a second opening in fluid communication with a second channel. In some embodiments, first opening or the second opening or both are dimensioned to accept a given droplet of the plurality of droplets.

In some embodiments, operation (b) comprises (i) directing the plurality of droplets along the first or second channel and (ii) providing a first liquid phase in the first channel and a second liquid phase in the second channel to retain the plurality of droplets in the plurality of wells. The first liquid phase and/or the second liquid phase of such embodiments may be individually or collectively immiscible with the droplet. In some embodiments, the first liquid phase is different that the second liquid phase.

In some embodiments the method may further comprise using at least one sensor in sensing communication with the plurality of wells to detect signals from the plurality of droplets. The signals may be indicative of the presence or absence of nucleic acid amplification product(s). In some embodiments the at least one sensor is an optical sensor and the signals are optical signals.

An additional aspect of the disclosure provides a method for processing a biological sample comprising: (a) providing a sample processing unit comprising a plurality of wells; (b) dispensing a plurality of droplets into the plurality of wells; (c) using a first fluid phase in the first channel and a second fluid phase in the second channel to confine the plurality of droplets in the plurality of wells; and (d) using at least one heating element to convert electrical energy or electromagnetic energy into thermal energy, subjecting the plurality of droplets to heating sufficient to conduct one or more nucleic acid amplification reactions on the biological sample or a portion thereof in the presence of reagents, thereby generating an amplification product(s) of the biological sample or a portion thereof.

In some embodiments, each of said plurality of wells is configured to receive and confine the plurality of droplets. The droplets may comprise the biological sample or a portion thereof or both. Each of the plurality of wells, in some embodiments, comprise a first opening in fluid communication with a first channel and a second opening in fluid communication with a second channel. The first opening or the second opening may be dimensioned to accept a given droplet of the plurality of droplets.

In some embodiments, the plurality of wells is in thermal communication with at least one heating element. The at least one heating element of some embodiments converts electrical or electromagnetic energy into thermal energy.

An additional aspect of the disclosure provides a system for processing a biological sample comprising: (1) a sample processing unit comprising (a) a plurality of wells configured to retain a plurality of droplets (themselves comprising the biological sample) during heating (wherein at least one heating element that converts electrical or electromagnetic energy into thermal energy is in thermal communication with the plurality of wells) and (b) a fluid flow path in fluid communication with the plurality of wells (each of the plurality of wells comprising a sidewall having a material mesh); and (2) a controller operatively coupled to said sample processing unit. In some embodiments, the controller is programmed to (i) direct a flow of the plurality of droplets through the fluid flow path to the plurality of wells, such that the plurality of droplets deposits in the plurality of wells; and (ii) use the at least one heating element to convert the electrical energy or electromagnetic energy into thermal energy and subject the plurality of droplets to heating, thereby processing the biological sample.

An additional aspect of the disclosure provides a system for performing a nucleic acid amplification reaction on a biological sample comprising: (1) a sample processing unit comprising a plurality of wells (each of the plurality of wells being configured to receive and confine at most a single droplet of a plurality of droplets comprising the biological sample, a portion thereof, or reagents necessary for a nucleic acid amplification reaction, or any combination thereof) in thermal communication with at least one heating element; and (2) a controller operatively coupled to the sample processing unit, wherein the controller is programmed to (i) direct dispensing of the plurality of droplets into the plurality of wells; and (ii) use the at least one heating element to convert the electrical energy or electromagnetic energy into thermal energy and subject the plurality of droplets to heating under conditions sufficient to conduct the nucleic acid amplification reaction on the biological sample or portion thereof in the presence of the reagents, to generate an amplification product(s) of the biological sample or portion thereof.

An additional aspect of the disclosure provides a system for processing a biological sample, the system comprising: (1) a sample processing unit comprising a plurality of wells (each of which is configured to receive and confine a plurality of droplets comprising the biological sample or a portion thereof), wherein each of the plurality of wells has a first opening in fluid communication with a first channel and a second opening in fluid communication with a second channel (the first or second opening or both may be dimensioned to accept a given droplet of said plurality of droplets), and wherein the plurality of wells is in thermal communication with at least one heating element (e.g., a heating element that converts electrical or electromagnetic energy into thermal energy); and (2) a controller (programmed to (i) direct the dispensing of the plurality of droplets into the plurality of wells, (ii) confine the plurality of droplets in the plurality of wells using a first fluid phase in the first channel and a second fluid phase in the second channel, and (iii) use at least one heating element to convert electrical energy or electromagnetic energy into thermal energy, heating the plurality of droplets under conditions sufficient to conduct at least one nucleic acid amplification reaction on the biological sample or portion thereof in the presence of said reagents, and thereby generating an amplification product(s) of the biological sample or portion thereof) operatively coupled to the sample processing unit.

An additional aspect of the disclosure provides an apparatus for processing a biological sample, the apparatus comprising at least one heating element (configured to convert electrical or electromagnetic energy into thermal energy) and a substrate (comprising a plurality of wells configured to retain a plurality of droplets of the biological sample or a portion thereof during heating). The plurality of wells of some embodiments is in thermal communication with at least one heating element. Furthermore, the plurality of wells may comprise a hygroscopic material to aid in retaining the plurality of droplets within the plurality of wells during heating.

An additional aspect of the disclosure provides an apparatus for processing a biological sample, the apparatus comprising at least one heating element (configured to convert electrical or electromagnetic energy into thermal energy) and a substrate (comprising a plurality of wells in thermal communication with at least one heating element). In some embodiments, the plurality of wells is configured to retain a plurality of droplets comprising the biological sample during heating of the plurality of droplets with the at least one heating element. Each of the plurality of wells, in some embodiments, comprises a sidewall having a material mesh.

An additional aspect of the disclosure provides an apparatus for processing a biological sample, the apparatus comprising at least one heating element (configured to convert electrical or electromagnetic energy into thermal energy) and a substrate comprising a plurality of wells configured to retain a plurality of droplets comprising the biological sample during heating (the plurality of wells being in thermal communication with at least one heating element). Each of the plurality of wells may have a first opening in fluid communication with a first channel and a second opening in fluid communication with a second channel. Either the first opening or the second opening, or both, may be dimensioned to accept a given droplet of the plurality of droplets.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 schematically illustrates an example configuration of a heating element;

FIG. 2 schematically illustrates an example configuration of a heating element;

FIGS. 3A and 3B schematically illustrate example populations of droplets;

FIG. 4 schematically illustrates example system and method for processing a biological sample;

FIG. 5 schematically illustrates an example system and method for processing a biological sample;

FIG. 6 schematically illustrates an example computer control system that is programmed or otherwise configured to implement methods provided herein;

FIGS. 7A-7C schematically illustrate a support system;

FIG. 8 schematically illustrates a temperature monitoring system comprising a plurality of temperature indicators;

FIG. 9 schematically illustrates a graph demonstrating the signal transmitted by a temperature indicator as a function of temperature;

FIG. 10 schematically illustrates a cross-sectional view of a temperature monitor comprising a support system; and

FIG. 11 schematically depicts an example heating device as described in Example 1; and

FIG. 12 graphically depicts temperature vs. time data obtained as part of Example 1.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular forms “a”, “an” and “the” include plural references unless the context clearly dictates otherwise. For example, the term “a molecule” includes a plurality of molecules, including mixtures thereof.

As used herein, the term “biological sample” generally refers to any sample containing or suspected of containing biological material, such as, for example, a protein (e.g., a protein molecule), a peptide (e.g., a peptide molecule), a nucleic acid (e.g., a nucleic acid molecule) , a lipid (e.g., a lipid molecule), a cell, a carbohydrate (e.g., a carbohydrate molecule), etc. A biological sample may comprise a bodily fluid, a body tissue or any combination thereof. Non-limiting examples of biological samples include feces, urine, sputum, saliva, blood, breast milk, plasma, breath, semen, cerebrospinal (CSF) fluid, mucus, lymph, earwax, hair, sweat, synovial fluid, vaginal discharge and oral mucosa. A biological sample may be directly obtained from its source without further processing or may be obtained from a subject and further processed (e.g., contacted with a lysis agent to lyse a cell in the biological sample, purified from a raw sample, etc.) for downstream reaction(s) and/or analysis. In some examples, a biological sample is obtained from a cell-free bodily fluid of a subject, such as whole blood. In some examples, a biological sample is an environmental sample (e.g., soil, waste, ambient air and etc.), industrial sample (e.g., samples from any industrial processes), a food sample (e.g., dairy products, vegetable products, and meat products). In some situations, a sample is obtained directly its source without further processing.

As used herein, the term “subject,” generally refers to an entity or a medium that has testable or detectable biological information. A biological sample can be obtained from a subject. A subject may be a person or an individual. A subject may be a vertebrate, such as, for example, a mammal. Non-limiting examples of mammals include murines, simians, humans, farm animals, sport animals, and pets. Other examples of subjects include, for example, food, plant, soil, and water. A subject may be a patient or an individual being treated or seeking treatment. A subject may be from a pathogen, such as a virus, bacterium, or microorganism. The target sequence may be from or correspond to a sequence of pathogen, such as a virus, bacterium or microorganism. Target sequences from and/or corresponding to a sequence from a virus may be from and/or correspond to an RNA virus or an DNA virus. In some embodiments, the virus from which a target sequence is taken or to which a target sequence corresponds is selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, Coxsackie virus, and Varicella virus. The influenza virus to which some target sequences correspond (and/or are taken from) include but are not limited to the group consisting of H1N1 virus, H3N2 virus, H7N9 virus and H5N1 virus. The adenovirus to which some target sequences correspond (and/or are taken from) may be adenovirus type 55 (ADV55) or adenovirus type 7 (ADV7). The hepatitis C virus to which some target sequences correspond (and/or are taken from) may be, for example, armored RNA-hepatitis C virus (RNA-HCV). The Coxsackie virus to which some target sequences correspond (and/or are taken from) includes Coxsackie virus A16.

A target sequence of some embodiment is from a pathogenic bacterium or a pathogenic protozoan. The pathogenic bacterium of such embodiments may be a gram-positive or gram-negative pathogenic bacterium. In some embodiments, the pathogenic bacterium is selected from the group consisting of Staphylococcus aureus, Listeria monocytogenes, Escherichia coli, Enterobacter sakazakii, Vibrio Parahemolyticus, and Shigella spp. In some embodiments, the pathogenic bacterium is Mycobacterium tuberculosis. In some embodiments, the pathogenic protozoan is Plasmodium. In some embodiments, the pathogenic bacterium is Salmonella.

As used herein, the terms “incubating” and “incubation” are used interchangeably and generally refer to keeping a sample, a mixture or a solution at certain temperature for a certain period of time, with or without shaking or stirring. An “incubation temperature” generally refers to a temperature at which incubation is permitted to occur. An “incubation time period” generally refers to an amount of time allotted for incubation to occur.

As used herein, the terms “denaturing” and “denaturation” are used interchangeably and generally refer to the full or partial unwinding of the helical structure of a double-stranded nucleic acid, and in some cases the unwinding of the secondary structure of a single stranded nucleic acid. Denaturation may include the inactivation of the cell wall(s) of a pathogen or the shell of a virus, and the inactivation of the protein(s) of inhibitors. Conditions at which denaturation may occur include a “denaturation temperature” that generally refers to a temperature at which denaturation is permitted to occur and a “denaturation duration” that generally refers to an amount of time allotted for denaturation to occur.

As used herein, the term “elongation” generally refers to the incorporation of nucleotides to a nucleic acid in a template directed fashion. Elongation may occur via the aid of an enzyme, such as, for example, a polymerase or reverse transcriptase. Conditions at which elongation may occur include an “elongation temperature” that generally refers to a temperature at which elongation is permitted to occur and an “elongation duration” that generally refers to an amount of time allotted for elongation to occur.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three dimensional structure, and may perform any function, known or unknown. Non-limiting examples of nucleic acids include DNA, RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product”. The term “reverse transcription amplification” generally refers to the generation of deoxyribonucleic acid (DNA) from a ribonucleic acid (RNA) template via the action of a reverse transcriptase. Moreover, a “nucleic acid amplification reaction” generally refers to a reaction or set of reactions in which a nucleic acid is subject to amplification.

Amplification of a nucleic acid may be linear, exponential, or any combination thereof. Non-limiting examples of nucleic acid amplification methods include reverse transcription, primer extension, ligase chain reaction (LCR), helicase-dependent amplification (e.g., amplification that is preceded by contacting the nucleic acid with a helicase), asymmetric amplification, rolling circle amplification, multiple displacement amplification (MDA), polymerase chain reaction (PCR) and variants thereof. Non-limiting examples of PCR variants include real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). In some cases, amplification is achieved with nested nucleic acid amplification. Moreover, amplification of a nucleic acid may be conducted isothermally or may be conducted via one or more temperature cycles (e.g., thermal cycling).

As used herein, the term “components necessary for conducting a chemical or biological reaction” generally refer to a material(s) that are required to complete and/or detect a given chemical or biological reaction on a biological sample. The components can be those necessary for conducting any type of chemical or biological reaction whose progress is initiated, sustained and/or enhanced with the inclusion of heat. Non-limiting examples include nucleic acid amplification reactions, denaturation reactions, cell lysis reactions, enzymatic reactions, reaction involving molecular recognition, and other chemical or biological reactions. Such components can include reactants, catalysts (e.g., enzymes), reaction mediums (e.g., buffer, solvent), reporter agents for reaction detection, and co-factors. Where the chemical or biological reaction is a nucleic acid amplification reaction, the components can be components necessary for the nucleic acid amplification reaction. Components necessary for a nucleic acid amplification reaction include one or more template nucleic acid molecules (e.g., a template nucleic acid molecule derived from a biological sample), one or more primers, one or more polymerizing enzymes, one or more deoxynucleotide triphosphates (dNTPs), co-factors (e.g., cations such as Mg²⁺) and a suitable reaction medium (e.g. buffer).

In some cases, the polymerizing enzyme is a polymerase (e.g., a DNA polymerase) that is capable incorporating nucleotides to a primer in a template directed manner. The polymerase may be any suitable polymerase and multiple polymerases may be implemented. Non-limiting examples of polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof.

As used herein, a “reporter agent” generally refers to a composition that yields a detectable signal, the presence or absence of which may be used to detect a chemical or biological reaction. In some cases, reporter agents may bind to initial reactants and changes in reporter agent levels may be used to detect amplified product. In some cases, reporter agents may only be detectable (or non-detectable) as a reaction progresses. A reporter agent may be an optically-active dye (e.g., a fluorescent dye). Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green H, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer HI, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methyl coumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some cases, a reporter agent may be a sequence-specific oligonucleotide probe that is optically active when hybridized with an amplified product. Due to sequence-specific binding of the probe to the amplified product, use of oligonucleotide probes can increase specificity and sensitivity of detection. A probe may be linked to any of the optically-active reporter agents (e.g., dyes) described herein and may also include a quencher capable of blocking the optical activity of an associated dye. Non-limiting examples of probes that may be useful used as reporter agents include TaqMan probes, TaqMan Tamara probes, TaqMan MGB probes, or Lion probes.

In some cases, a reporter agent may be an RNA oligonucleotide probe that includes an optically-active dye (e.g., fluorescent dye) and a quencher positioned adjacently on the probe. The close proximity of the dye with the quencher can block the optical activity of the dye. The probe may bind to a target nucleic acid sequence to be amplified. Upon the breakdown of the probe with the exonuclease activity of a DNA polymerase during amplification, the quencher and dye are separated, and the free dye regains its optical activity that can subsequently be detected.

As used herein, the term “fluid” generally refers to a liquid or a gas. A fluid cannot maintain a defined shape and will flow during an observable time frame to fill a container in which it is put. Thus, a fluid may have any suitable viscosity that permits flow. If two or more fluids are present, each fluid may be independently selected among essentially any fluids (liquids, gases, and the like).

As used herein, the term “aqueous phase” generally refers to a fluid phase that is made with, of, or from water, or a fluid that contains water. For example, an aqueous phase may be an aqueous solution with water as the solvent. An aqueous phase of the present disclosure may comprise reagents necessary for conducting a desired chemical reaction, e.g., a nucleic acid amplification reaction, such as polymerase chain reaction (PCR). Non-limiting examples of aqueous phases include, but are not limited to, water and other aqueous solutions comprising water, such as cell or biological medium, ethanol, salt solutions, etc.

As used herein, the term “continuous phase” generally refers to a fluid phase that forms a continuous flow. A continuous phase may be a fluid immiscible with an aqueous solution. For example, a continuous phase may be a non-aqueous fluid made from, with, or using a liquid other than water. Non-limiting examples of continuous phases include, but are not limited to, oils such as hydrocarbons, silicon oils, fluorinated oils (e.g., fluorocarbon oils, HFE 7100, HFE 7500, FC-40, FC-43, FC-70, FC-3208) and an organic solvent. In some cases, a continuous phase comprises a surfactant, such as a fluorosurfactant. The surfactant may comprise a hydrophobic tail and a hydrophilic head group, a polymer-based tail and a hydrophilic head group, a polymer-based tail and a polymer-based head group, a fluorinated tail and a hydrophilic head group, or a fluorinated polymer-based tail and a hydrophilic polymer-based head group. In some cases, the surfactant is of a di-block copolymer or tri-block copolymer type. For example, the surfactant may be a block copolymer, such as a tri-block copolymer consisting of two perfluoropolyether blocks and one poly(ethylene)glycol block. Other examples of surfactants include PFPE-PEG-PFPE (perfluoropolyether-polyethylene glycol-perfluoropolyether), tri-block copolymer EA-surfactant (RainDance Technologies) and DMP (dimorpholino phosphate)-surfactant (Baret, Kleinschmidt, et al., 2009).

As used herein, the terms “channel” or “flow channel” generally refer to a path that confines and/or directs the flow of a fluid. A channel of the present disclosure may be of any suitable length. The channel may be straight, substantially straight, or it may contain one or more curves, bends, etc. For example, the channel may have a serpentine or a spiral configuration. In some situations, the channel includes one or more branches, with some or all of which connected with one or more other channel(s). In some cases, a channel may be a microfluidic channel of a microfluidic device or system.

As used herein, the term “droplet” generally refers to an isolated portion of a first fluid (e.g., an aqueous phase) that is surrounded by a second fluid (e.g., a continuous phase), such as an emulsion comprising a dispersion of droplets of a first fluid in a second fluid. The first fluid may be substantially immiscible (or completely immiscible) with the second fluid. A droplet of the present disclosure may be spherical or assume other shapes, such as, for example, shapes with elliptical cross-sections. The diameter of a droplet, in a non-spherical droplet, is the diameter of a perfect mathematical sphere having the same volume as the non-spherical droplet. A droplet may include a skin. The skin may form upon heating the droplet. The skin may have a higher viscosity than an interior of the droplet. In some cases, the skin may prevent the droplet from fusing with other droplets. The droplets may include detectable moieties that permit detection of any signals generated from the biological and/or chemical reactions (e.g., nucleic acid amplification reactions). For example, the detectable moieties may yield a detectable signal whose presence or absence is indicative of a presence of an amplified product. The intensity of the detectable signal may be proportional to the amount of amplified product. In some embodiments, where amplified product is generated of a different type of nucleic acid than the target nucleic acid initially amplified, the intensity of the detectable signal may be proportional to the amount of target nucleic acid initially amplified. For example, in the case of amplifying a target RNA via parallel reverse transcription and amplification of the DNA obtained from reverse transcription, reagents necessary for both reactions may also comprise a detectable moiety that yield a detectable signal indicative of the presence of the amplified DNA product and/or the target RNA amplified. The intensity of the detectable signal may be proportional to the amount of the amplified DNA product and/or the original target RNA amplified. The use of a detectable moiety also enables real-time amplification methods, including real-time PCR for DNA amplification.

Detectable moieties may be linked with nucleic acids, including amplified products, by covalent or non-covalent interactions. Non-limiting examples of non-covalent interactions include ionic interactions, Van der Waals forces, hydrophobic interactions, hydrogen bonding, and combinations thereof. In some embodiments, detectable moieties bind to initial reactants and changes in detectable moiety levels are used to detect amplified product. In some embodiments, detectable moieties are only detectable (or non-detectable) as nucleic acid amplification progresses. In some embodiments, an optically-active dye (e.g., a fluorescent dye) is used as a detectable moiety. Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some embodiments, a detectable moiety is a sequence-specific oligonucleotide probe that is optically active when hybridized with an amplified product. Due to sequence-specific binding of the probe to the amplified product, use of oligonucleotide probes can increase specificity and sensitivity of detection. A probe may be linked to any of the optically-active detectable moieties (e.g., dyes) described herein and may also include a quencher capable of blocking the optical activity of an associated dye. Non-limiting examples of probes that may be useful as detectable moieties include TaqMan probes, TaqMan Tamara probes, TaqMan MGB probes, or Lion probes.

In some embodiments and where a detectable moiety is an RNA oligonucleotide probe that includes an optically-active dye (e.g., fluorescent dye) and a quencher positioned adjacently on the probe. The close proximity of the dye with the quencher can block the optical activity of the dye. The probe may bind to a target sequence to be amplified. Upon the breakdown of the probe with the exonuclease activity of a DNA polymerase during amplification, the quencher and dye are separated, and the free dye regains its optical activity that can subsequently be detected.

In some embodiments, a detectable moiety is a molecular beacon. A molecular beacon includes, for example, a quencher linked at one end of an oligonucleotide in a hairpin conformation. At the other end of the oligonucleotide is an optically active dye, such as, for example, a fluorescent dye. In the hairpin configuration, the optically-active dye and quencher are brought in close enough proximity such that the quencher is capable of blocking the optical activity of the dye. Upon hybridizing with amplified product, however, the oligonucleotide assumes a linear conformation and hybridizes with a target sequence on the amplified product. Linearization of the oligonucleotide results in separation of the optically-active dye and quencher, such that the optical activity is restored and may be detected. The sequence specificity of the molecular beacon for a target sequence on the amplified product can improve specificity and sensitivity of detection.

In some embodiments, a detectable moiety is a radioactive species. Non-limiting examples of radioactive species include ¹⁴C, ¹²³I, ¹²⁴I, ¹²⁵I, ¹³¹I, Tc99m, ³⁵S, and ³H.

In some embodiments, a detectable moiety is an enzyme that is capable of generating a detectable signal. Detectable signal may be produced by activity of the enzyme with its substrate or a particular substrate in the case the enzyme has multiple substrates. Non-limiting examples of enzymes that may be used as detectable moieties include alkaline phosphatase, horseradish peroxidase, I²-galactosidase, alkaline phosphatase, β-galactosidase, acetylcholinesterase, and luciferase.

In some embodiments, a detectable moiety may comprise a thermal liquid crystal (TLC), also known as a thermochromic liquid crystal, whose color response is a function of temperature. A TLC may comprise a material that changes its reflected color as a function of temperature when illuminated by a light of a first color (e.g., white, infrared, red, orange, yellow, green, blue, violet, ultraviolet). A detectable moiety comprising at least one TLC may reflect light (either visible or invisible) of a first wavelength at a first temperature and reflect light (either visible or invisible) of a second wavelength at a second temperature. In some embodiments, the detectable moiety may be disposed within a strip, a panel, a sheet, a plate, or a sticker. In some embodiments, the detectable moiety may be disposed within at least one droplet (in some embodiments selected from a plurality of droplets) disposed within a system such that the temperature of sample may be measured by detecting the color of the at least one droplet. Detection of the detectable moiety disposed within at least one droplet may be used to calibrate the system (e.g., prompting the controller to direct heat generation and/or cooling, prompting the controller to generate the amount droplets or the rate of droplet generation, etc.).

As used herein, the term “thermal communication” generally refers to a state in which two or more materials are capable of exchange energy, such as thermal energy, with one another. Such exchange of energy may be by way of transfer of energy from one material to another material. Such transfer of energy may be radiative, conductive, or convective heat transfer. The energy may be thermal energy. In some examples, two or more materials that are in thermal communication with one another are in thermal contact with one another, such as, for example, direct physical contact or contact through one or more intermediary materials.

As used herein, the term “vessel” generally refers to a container that can compartmentalize matter. For example, a vessel may be configured to contain fluid matter, such as a solution. In some cases, a vessel may be configured to contain a plurality of partitions (e.g., a plurality of wells, a plurality of droplets in an emulsion). Non-limiting examples of vessels include a tube (e.g., a test tube, a centrifuge tube), a cuvette, a chamber, a beaker, a reservoir, a flow channel (e.g., a flow channel of a fluidic device), a capillary tube, a well, a multi-well plate, a bottle and a flask. A vessel may be constructed of any suitable type of material, with non-limiting examples of such materials including glass, elemental metals, metal alloys, plastic, laminate, ceramics or any combination thereof.

A vessel may have any suitable volumetric capacity. In some cases, the volumetric capacity of a vessel may be relatively low to accommodate small biological sample sizes, while minimizing excess space that can result in greater energy requirements during heating. For example, a volumetric capacity of a vessel may be at most about 10 milliliters (mL), at most about 7 mL, at most about 5 mL, at most about 2 mL, at most about 1 mL, at most about 0.5 mL, at most about 0.1 mL, at most about 0.05 mL, at most about 0.01 mL, at most about 0.005 mL, at most about 0.001 mL or less. In some cases, the volumetric capacity of a vessel may be maximized to accommodate a large range of biological sample sizes. In some cases, a volumetric capacity of a vessel may be at least about 0.001 mL, at least about 0.005 mL, at least about 0.01 mL, at least about 0.05 mL, at least about 0.1 mL, at least about 0.5 mL, at least about 1 mL, at least about 2 mL, at least about 5 mL, at least about 7 mL, at least about 10 mL or more.

As used herein, the term “electromagnet” refers to a device, system component or material capable of generating a magnetic field upon electrical current flow through one or more components of the device or system component or through the material. An electromagnet may comprise one or more coils (e.g., wire coils, metal coils) through which electrical current can flow. In some cases, the coil may be wrapped around a core, such as a ferromagnetic or ferrimagnetic core. Upon flow of electrical current a magnetic field is generated. The electrical current may be direct current (DC) or alternating current (AC). When alternating current is supplied to an electromagnet, an alternating magnetic field can be generated by the electromagnet. Moreover, an electromagnet can be in electrical communication with circuitry that provides electrical current to the electromagnet. Such circuitry can be controlled by a computer control system, including an example control system described elsewhere herein. Furthermore, at least a portion of an electromagnet may be shielded in order to minimize interference from the surrounding environment. For example, a Faraday cage may be used.

As used herein, the term “heating element” generally refers to a material in which heat is generated and can be utilized or transferred to another medium, such as a surrounding medium. In some cases, a heating element generates heat upon flow of eddy current through at least a portion, all or substantially all of the heating element. Materials comprising such a heating element include those comprising free electrons. Eddy currents can be induced to flow in a heating element via magnetic induction, such as electromagnetic induction (e.g., via coupling the heating element to a magnetic field generated by an electrical current). Accordingly, the term “inductive heating”, as used herein, generally refers to heating with the aid of electrical current generated in a heating element in the presence of a magnetic field. Moreover, the term “inductive coupling” generally refers to the coupling of a heating element to a magnetic field, whereby the magnetic field inductively generates electrical current in the heating element. In some cases, the heating element is a thermoelectric material, such as indium tin oxide or a variant. A thermoelectric electric material is any wherein a thermoelectric effect (e.g., the Seebeck effect, the Peltier effect, the Thomson effect, etc.) may be made manifest strongly, conveniently, or actively in the material. For example, a thermoelectric material may convert electrical (or electromagnetic) energy (e.g., a change in electrical potential, an injection of current, etc.) into thermal energy. For example, a thermoelectric heating element may comprise a resistive heating element. Thermoelectric materials that may comprise the heating element include but are not limited to bismuth chalcogenides (e.g., Bi₂Te₃, Bi₂Se₃), clathrates (e.g., inorganic clathrates), electrically conducting materials (e.g., electrically conducting organic materials), half heusler alloys, lead telluride (e.g., thallium-doped PbTe, sodium-doped PbTe, etc., magnesium group IV compounds (e.g., Mg₂B^{IV}, where B^{IV} can be Si, Ge, Sn), oxide thermoelectrics, silicides, skutterudite thermoelectrics, sodium cobaltate, and tin selenide, their variants and their alloys. The heating element may be an optically clear heating element such as indium tin oxide. Optically clear heating elements may be layered (coated) atop a substrate such as a glass, a plastic, a metal, an organic material, etc. One or more heating elements may comprise a resistance heater, a positive temperature coefficient (PTC) heater, a negative temperature coefficient (NTC) heater, a high temperature thick film, or a high resistant thick film. Heating elements may take the form of particles, strips, pads, wells, walls, vessels, plates, sleeves, rods, cones, wires, films, etc. In some case, the heating element is a transparent conductive film. The heating element may be formed of a polymeric material, a semi-conducting material, or an insulating material. For example, the heating element is formed of polyacetylene, polyaniline, polypyrrole or polythiophenes, poly(3,4-ethylenedioxythiophene) (PEDOT), poly(4,4-dioctylcyclopentadithiophene), a combination or variant thereof. As another example, the heating element is formed of indium tin oxide (ITO). The heating element may be formed of other transparent conducting films in addition to those comprising indium tin oxide, such as those comprising fluorine doped tin oxide, those comprising doped zinc oxide, those comprising carbon (such as those comprising carbon nanotube networks, graphene, etc.), those comprising poly(3,4-ethylenedioxythiophene), etc.

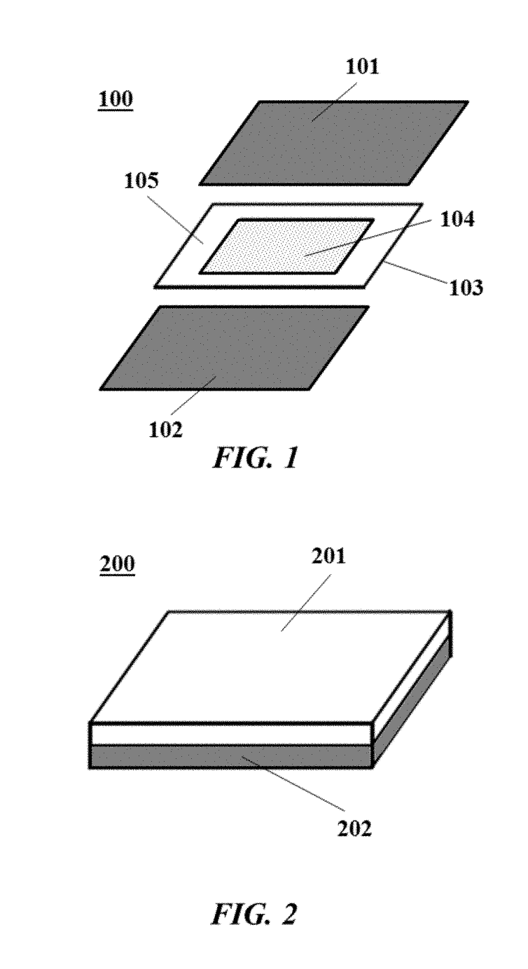

In some cases, a heating element may be a component of a vessel. For example, the heating element may be integrated into a structural component of a vessel, such as a vessel wall or lid. Examples of such components are schematically shown in FIG. 1 and FIG. 2. FIG. 1 is an exploded view of an example vessel component 100 (e.g., a wall and/or a lid), which comprises top 101 and bottom 102 laminate layers. Each of the top laminate layer 101 and bottom laminate layer 102 comprises a laminate material. Positioned in-between the top laminate layer 101 and bottom laminate layer 102 is a heating layer 103. The heating layer 103 comprises a second region 104 comprising one or more heating elements. The one or more heating elements may be particles. A second region 105 circumscribes the first region 104. In some cases, at least a portion of the second region (on either of both of its top and bottom sides) comprises an adhesive material that couples the top laminate layer 101 and bottom laminate layer 102 to the heating layer 103. When used as a structural component of a vessel, the heating layer 104 (and, thus, its heating element(s) does not make physical contact with an outside environment due to its layering between the top laminate layer 101 and the bottom laminate layer 102.

With reference to FIG. 2, an example vessel component 200 (e.g., a wall and/or a lid), which is shown in a layered view, comprises laminate layer 201 and heater layer 202 comprises a laminate material. The laminate layer 201 comprises a laminate material, whereas the heater layer 202 comprises one or more heating elements. In some cases, heater layer 202 comprises a single heating element that comprises a single material. In some cases, heater layer 202 comprises multiple heating elements comprising a single material. In some cases, heater layer 202 comprises a single heating element that comprises multiple materials. In some cases, heater layer 202 comprises multiple heating elements that may individually or collectively comprise multiple materials. In some cases, at least a portion of the bottom side of the laminate layer 201 and the heater layer 202 are physically joined together via an adhesive material that adheres to the surface. The laminate layer 201 and the heater layer 202 may be mechanically coupled (such being stitched together, welded together, soldered, etc.). When used as a structural component of a vessel, the heating layer 202 (and, thus, its heating element(s) may make physical contact with an outside environment due to its exposed surface opposite the laminate layer 201.

A heating element can include or be formed of any suitable material(s) with non-limiting examples that include carbon, iron, copper, aluminum, chromium, nickel, variants of the aforementioned, alloys of the aforementioned, composites of the aforementioned, or any combination thereof (e.g., steel), ferromagnetic materials, and paramagnetic materials. Suitable materials may include other conducting materials, such as conducting polymeric materials (e.g., polymers doped with iron), conducting ceramic materials (e.g., indium tin oxide), or thermoelectric materials. Moreover, a heating element may comprise a magnetic material (e.g., a ferromagnetic material, a paramagnetic material, a superparamagnetic material, etc.). In some cases, a heating element comprises a polymer and at a magnetically-active material supported by the polymer, where the magnetically-active material generates heat upon inductive coupling to the magnetic field. Furthermore, a heating element may comprise any suitable shape, size or dimension. For example, a heating element that is a particle may be regularly shaped (e.g., spherical shaped, rod shaped, ellipsoid shaped, star-shaped, tubular shaped, triangularly shaped, etc.) or may be irregularly shaped. A heating element in the form or a particle can be of any suitable size, including a nanometer scale particle (e.g., nanoparticle), a micrometer scale (e.g., a microparticle) and larger particles. Moreover, the particle, for example, is a spherical particle (e.g., a nanosphere, a microsphere), a tubular particle (e.g., a nanotube), a wire-shaped particle (e.g., a nanowire), a rod-shaped particle (e.g., a nanorod), is a another regularly shaped particle, or is irregularly shaped. In some cases, a heating element may have a dimension (e.g., diameter) that is at most about 500 micrometers (μm), at most about 100 μm, at most about 50 μm, at most about 10 μm, at most about 5 μm, at most about 1 μm, at most about 500 nanometers (nm), at most about 100 nm, at most about 50 nm, at most about 10 nm, at most about 5 nm, at most about 1 nm or less. By way of another non-limiting example, a heating element that is a sheet may be regularly shaped (e.g., having area demarcated by a square, rectangle, triangle, circle, oval, etc.), have a narrow cross section (such as a thin strip), or it may irregularly shaped. A heating element in the form of a sheet may be of any suitable size including having a thickness of no greater than about 2 mm, 1 mm, 900 micrometers (μm), 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, or 1 μm.

The present disclosure provides systems and methods for processing a biological sample. In particular, the systems and methods may include or make use of inductive heating that can be used to heat a biological sample. The biological sample, or a portion thereof, may be a component of a solution and/or may be included in a partition such as a droplet in an emulsion or a well in a multi-well device. In some cases, the biological sample, or a portion thereof, may be in thermal contact with one or more heating elements. Such heating elements can include materials that have a free electron and/or are materials in which eddy currents are generated when coupled to a magnetic field. Such eddy currents generate heat that can be used for heating a biological sample.

In an aspect, the disclosure provides a method for processing a biological sample. The method includes: (a) providing a solution comprising the biological sample and one or more heating elements in a vessel. The one or more heating elements can be dispersed in the solution and generate heat upon inductive coupling to a magnetic field. The method also includes (b) bringing the solution in contact with the magnetic field; and (c) subjecting the solution comprising the biological sample to heating upon inductive coupling of the one or more heating elements to the magnetic field.

In some cases, the method may further comprise subjecting the solution to heating with one or more heating elements that are components of the vessel (e.g., a wall component of a vessel). Such heating is achieved upon inductive coupling of the relevant component(s) of the vessel to the magnetic field. In some cases, the method further comprises providing the magnetic field through a permanent magnet and/or an electromagnet. Examples of electromagnets and their operation are described elsewhere herein. Moreover, in some cases, (a) and (b) are performed simultaneously.

In some cases, the method further comprises (d) subjecting the solution to cooling. Subjecting the solution to cooling can occur upon decoupling the one or more heating elements from the magnetic field and/or by positioning the solution in a cooling zone comprising one or more cooling units (e.g., a wind tunnel, a convective cooling unit, a cooling block, an evaporative cooling unit, a thermoelectric device (e.g., a Peltier device, indium tin oxide, etc.), a cooling bath unit) that are in thermal communication with the solution. Subjecting the solution to cooling can be achieved via any suitable route of energy transfer, including convective and conductive cooling routes. In some cases, the method further comprises repeating (b)-(d), thereby subjecting the solution to repeated heating and cooling (e.g., thermal cycling). Thermal cycling of the solution can be useful for a host of sample processing and/or biological/chemical reactions, including preparation of the biological sample for a nucleic acid amplification reaction and conducting the nucleic acid amplification reaction.

In some cases, the solution comprises components necessary for conducting a chemical or biological reaction on the biological sample. For example, the biological sample may comprise a nucleic acid molecule and the solution may comprise components necessary for a nucleic acid amplification reaction, with examples of nucleic acid amplification reactions and necessary components for nucleic acid amplification provided elsewhere herein. Where the solution comprises components necessary for conducting a chemical or biological reaction on the biological sample, the method may further comprise conducting the chemical or biological reaction on the biological sample (e.g., conducting a nucleic acid amplification reaction(s)). Moreover, the method may also comprise detecting one or more signals indicative of the chemical or biological reaction. Any suitable detector and detection modality can be used, including examples of such provided elsewhere herein.

In another aspect, the disclosure provides a system for processing a biological sample. The system comprises a vessel that holds a solution comprising the biological sample and one or more heating elements. The one or more heating elements generate heat upon inductive coupling to a magnetic field. The system also comprises a magnetic field application unit that provides a magnetic field to the solution; and a controller operatively coupled to the magnetic field application unit. The controller is programmed to direct the magnetic field application unit to apply the magnetic field to the solution, thereby subjecting the solution to heating upon inductive coupling of the one or more heating elements to the magnetic field. The controller may comprise one or more computer processors associated with a computer control system. Examples of computer control systems are provided elsewhere herein. In some cases, the vessel may itself comprise one or more components (e.g., wall components) that comprise one or more heating elements that generate heat upon inductive coupling of the component(s) to the magnetic field.

In some cases, the magnetic field application unit comprises a permanent magnet and/or an electromagnet. The permanent magnet and/or electromagnet can provide a magnetic field to the solution. Moreover, the system can further comprise a cooling zone that subjects the solution to cooling. A cooling unit (e.g., a wind tunnel, a convective cooling unit, a cooling block, an evaporative cooling unit, a Peltier device, a cooling bath unit) may provide cooling to the cooling zone. Moreover, the controller may comprise one or more computer processors that are individually or collectively programmed to alternately and sequentially position the solution in the magnetic field and in the cooling zone. For example, the vessel may be rotatable with respect to the magnetic field application unit and/or the cooling zone, or vice versa. The magnetic field (and, perhaps, the magnetic field application unit) and cooling zone may be angularly separated such that rotation of the vessel about an axis of rotation passes the solution from one of the magnetic field and the cooling zone to the other of the two. In some cases, rotation of the magnetic field application unit and/or the cooling zone about an axis of rotation exposes the vessel (and, thus, any contained solution) to cyclical heating (and cooling where applicable).

In some cases, the system may further comprise a detector. The detector may be positioned adjacent to the vessel or may be positioned at a distance from said vessel. The detector can detect, during detection, one or more signals from the solution that are indicative of a chemical or biological reaction on the biological sample. Any suitable detector and detection modality may be used, including examples of detectors and detection modalities described elsewhere herein.

In various aspects, a method or system for processing a biological sample comprises a solution or providing a solution comprising one or more heating elements. In some cases, the one or more heating elements are floating or suspended in the solution (e.g., in the case of heating elements comprising insoluble materials). In some cases, at least a subset of the one or more heating elements is dissolved in the solution. Moreover, the solution may comprise one or more droplets, such as droplets in an emulsion. Examples of emulsions are described elsewhere herein. In some cases, at least a subset of the one or more heating elements is in a given one of the one or more droplets. In some cases, the solution comprises components necessary for conducting a chemical or biological reaction on the biological sample. In some cases, the chemical or biological reaction is a nucleic acid amplification reaction. The components may be aliquoted across the one or more droplets. In some cases, the one or more heating elements may be co-aliquoted with the components (e.g., droplets comprise components and heating element and biological sample (or a portion thereof)) or may be aliquoted separate from the components (e.g., droplets comprise components and biological sample (or a portion thereof), separate droplets comprise heating element).

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration.

Denaturation temperatures may vary depending upon, for example, the particular biological sample analyzed, the particular source of target nucleic acid (e.g., viral particle, bacteria) in the biological sample, the reagents used, and/or the desired reaction conditions. For example, a denaturation temperature may be from about 80° C. to about 110° C. In some examples, a denaturation temperature may be from about 90° C. to about 100° C. In some examples, a denaturation temperature may be from about 90° C. to about 97° C. In some examples, a denaturation temperature may be from about 92° C. to about 95° C. In still other examples, a denaturation temperature may be about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

Denaturation durations may vary depending upon, for example, the particular biological sample analyzed, the particular source of target nucleic acid (e.g., viral particle, bacteria) in the biological sample, the reagents used, and/or the desired reaction conditions. For example, a denaturation duration may be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, a denaturation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

Elongation temperatures may vary depending upon, for example, the particular biological sample analyzed, the particular source of target nucleic acid (e.g., viral particle, bacteria) in the biological sample, the reagents used, and/or the desired reaction conditions. For example, an elongation temperature may be from about 30° C. to about 80° C. In some examples, an elongation temperature may be from about 35° C. to about 72° C. In some examples, an elongation temperature may be from about 45° C. to about 65° C. In some examples, an elongation temperature may be from about 35° C. to about 65° C. In some examples, an elongation temperature may be from about 40° C. to about 60° C. In some examples, an elongation temperature may be from about 50° C. to about 60° C. In still other examples, an elongation temperature may be about 35°, 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

Elongation durations may vary depending upon, for example, the particular biological sample analyzed, the particular source of target nucleic acid (e.g., viral particle, bacteria) in the biological sample, the reagents used, and/or the desired reaction conditions. For example, an elongation duration may be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, an elongation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) necessary to obtain a detectable amplified product (e.g., a detectable amount of amplified DNA product that is indicative of the presence of a target RNA in a biological sample). For example, the number of cycles necessary to obtain a detectable amplified product (e.g., a detectable amount of DNA product that is indicative of the presence of a target RNA in a biological sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target RNA in a biological sample) may be obtained at a cycle threshold value (Ct) of less than 100, 75, 70, 65, 60, 55, 50, 45, 40, 35, 30, 25, 20, 15, 10, or 5.

The time for which amplification yields a detectable amount of amplified product indicative of the presence of a target nucleic acid amplified can vary depending upon the biological sample from which the target nucleic acid was obtained, the particular nucleic acid amplification reactions to be conducted, and the particular number of cycles of amplification reaction desired. For example, amplification of a target nucleic acid may yield a detectable amount of amplified product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a target RNA may yield a detectable amount of amplified DNA product indicative to the presence of the target RNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, a reaction mixture may be subjected to a plurality of series of primer extension reactions. An individual series of the plurality may comprise multiple cycles of a particular primer extension reaction, characterized, for example, by particular denaturation and elongation conditions as described elsewhere herein. Generally, each individual series differs from at least one other individual series in the plurality with respect to, for example, a denaturation condition and/or elongation condition. An individual series may differ from another individual series in a plurality of series, for example, with respect to any one, two, three, or all four of denaturing temperature, denaturing duration, elongation temperature, and elongation duration. Moreover, a plurality of series may comprise any number of individual series such as, for example, at least about or about 2, 3, 4, 5, 6, 7, 8, 9, 10, or more individual series.

For example, a plurality of series of primer extension reactions may comprise a first series and a second series. The first series, for example, may comprise more than ten cycles of a primer extension reaction, where each cycle of the first series comprises (i) incubating a reaction mixture at about 92° C. to about 95° C. for no more than 30 seconds followed by (ii) incubating the reaction mixture at about 35° C. to about 65° C. for no more than about one minute. The second series, for example, may comprise more than ten cycles of a primer extension reaction, where each cycle of the second series comprises (i) incubating the reaction mixture at about 92° C. to about 95° C. for no more than 30 seconds followed by (ii) incubating the reaction mixture at about 40° C. to about 60° C. for no more than about 1 minute. In this particular example, the first and second series differ in their elongation temperature condition. The example, however, is not meant to be limiting as any combination of different elongation and denaturing conditions may be used.

In some embodiments, the ramping time (i.e., the time the thermal cycler takes to transition from one temperature to another) and/or ramping rate can be important factors in amplification. For example, the temperature and time for which amplification yields a detectable amount of amplified product indicative of the presence of a target nucleic acid can vary depending upon the ramping rate and/or ramping time. The ramping rate can impact the temperature(s) and time(s) used for amplification.

In some cases, the ramping time and/or ramping rate can be different between cycles. In some situations, however, the ramping time and/or ramping rate between cycles can be the same. The ramping time and/or ramping rate can be adjusted based on the sample(s) that are being processed.

In some situations, the ramping time between different temperatures can be determined, for example, based on the nature of the sample and the reaction conditions. The exact temperature and incubation time can also be determined based on the nature of the sample and the reaction conditions. In some embodiments, a single sample can be processed (e.g., subjected to amplification conditions) multiple times using multiple thermal cycles, with each thermal cycle differing for example by the ramping time, temperature, and/or incubation time. The best or optimum thermal cycle can then be chosen for that particular sample. This provides a robust and efficient method of tailoring the thermal cycles to the specific sample or combination of samples being tested.

In some embodiments, a target nucleic acid may be subjected to a denaturing condition prior to initiation of a primer extension reaction. In the case of a plurality of series of primer extension reactions, the target nucleic acid may be subjected to a denaturing condition prior to executing the plurality of series or may be subjected to a denaturing condition between series of the plurality. For example, the target nucleic acid may be subjected to a denaturing condition between a first series and a second series of a plurality of series. Non-limiting examples of such denaturing conditions include a denaturing temperature profile (e.g., one or more denaturing temperatures) and a denaturing agent.

An advantage of conducting a plurality of series of primer extension reaction may be that, when compared to a single series of primer extension reactions under comparable denaturing and elongation conditions, the plurality of series approach yields a detectable amount of amplified product that is indicative of the presence of a target nucleic acid in a biological sample with a lower cycle threshold value. Use of a plurality of series of primer extension reactions may reduce such cycle threshold values by at least about or about 1%, 5%, 10%, 15%, 20%, 25%, 30%, 35%, 40%, 45%, 50%, 55%, 60%, 65%, 70%, 75%, 80%, 85%, 90%, or 95% when compared to a single series under comparable denaturing and elongation conditions.

In some embodiments, a biological sample may be preheated prior to conducting a primer extension reaction. The temperature (e.g., a preheating temperature) at which and duration (e.g., a preheating duration) for which a biological sample is preheated may vary depending upon, for example, the particular biological sample being analyzed. In some examples, a biological sample may be preheated for no more than about 60 minutes, 50 minutes, 40 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, 45 seconds, 30 seconds, 20 seconds, 15 seconds, 10 seconds, or 5 seconds. In some examples, a biological sample may be preheated at a temperature from about 80° C. to about 110° C. In some examples, a biological sample may be preheated at a temperature from about 90° C. to about 100° C. In some examples, a biological sample may be preheated at a temperature from about 90° C. to about 97° C. In some examples, a biological sample may be preheated at a temperature from about 92° C. to about 95° C. In still other examples, a biological sample may be preheated at a temperature of about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

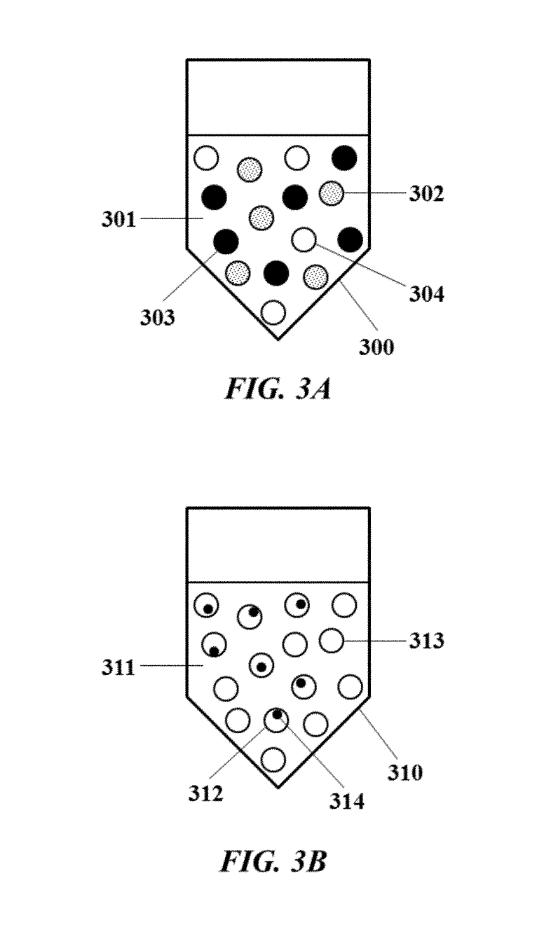

Examples of two populations of droplets are schematically shown in FIG. 3A and FIG. 3B. As shown in FIG. 3A, a vessel 300 comprises a continuous phase 301 comprising a population of droplets. The population of droplets comprises three types of droplets: reaction droplets 302, heating droplets 303 and empty droplets 304. The reaction droplets 302 comprise a portion of a biological sample and components necessary for conducting a chemical or biological reaction on the biological sample. As an alternative, the reaction droplets 302 include the entirety of the biological sample. The heating droplets 303 comprise one or more heating elements, without biological sample or components necessary for conducting the chemical or biological reaction. Upon inductive coupling of the heating droplets 303 (and, thus, contained heating elements) to a magnetic field, heat is generated in the heating elements, which then flows to the reaction droplets 302.

The population of droplets may comprise a fourth type of droplet containing a detectable moiety. The detectable moiety of the fourth type of droplet, referred to here as a “monitoring” type of droplet, may comprise a detectable moiety capable of detecting temperature, temperature differences, heat, heat flux, or a thermal dose, or any combination thereof. For example, monitoring droplets may comprise a type of thermal liquid crystal (e.g., a nanoparticle of a thermal liquid crystal) that reflects a light having a first wavelength at a first temperature and may reflect light having a second wavelength at a second temperature, thereby monitoring temperature of the monitoring droplet. In some embodiments, a signal from the monitoring droplets may be indicative of a state of the population of droplets. For example, the signal from one or more monitoring droplets may indicate the temperature of the solution, the temperature of the vessel, the temperature of at least a subset of the population of droplets (e.g., nearest neighboring droplets, droplets of a first type, droplets of a second type, droplets of a third type, etc.), or any combination thereof.

As shown in FIG. 3B, a vessel 310 comprises a continuous phase 311 comprising a population of droplets. The population of droplets comprises two types of droplets: reaction droplets 312 and empty droplets 313. The reaction droplets 312 comprise a portion of a biological sample, components necessary for conducting a chemical or biological reaction on the biological sample, and one or more heating elements 314. As an alternative, the reaction droplets 312 include the entirety of the biological sample. Upon inductive coupling of the reaction droplets 312 (and, thus, contained heating elements 314) to a magnetic field, heat is generated in the heating elements 314 and is available for the chemical or biological reaction in the reaction droplets 312.

In various aspects, a solution can have any suitable volume. In some cases, the volume of a solution may be kept relatively low in order to, for example, accommodate small sample sizes and/or permit faster processing times. For example, the volume of a solution may be at most about 100 mL, at most about 50 mL, at most about 10 mL, at most about 9 mL, at most about 8 mL, at most about 7 mL, at most about 6 mL, at most about 5 mL, at most about 4 mL, at most about 3 mL, at most about 2 mL, at most about 1 mL, at most about 0.7 mL, at most about 0.5 mL, at most about 0.3 mL, at most about 0.1 mL, at most about 0.05 mL, at most about 0.01 mL, at most about 0.005 mL, at most about 0.001 mL or less. In some cases, the volume of a solution may be maximized in order to, for example, accommodate large sample sizes without separate processing. For example, the volume of a solution may be at least about 0.001 mL, at least about 0.005 mL, at least about 0.01 mL, at least about 0.05 mL, at least about 0.1 mL, at least about 0.3 mL, at least about 0.5 mL, at least about 0.7 mL, at least about 1 mL, at least about 2 mL, at least about 3 mL, at least about 4 mL, at least about 5 mL, at least about 6 mL, at least about 7 mL, at least about 8 mL, at least about 9 mL, at least about 10 mL, at least about 50 mL, at least about 100 mL or more.

In another aspect, the disclosure provides a method for processing a biological sample. The method comprises (a) providing a plurality of partitions, where at least a subset of the plurality of partitions comprises the biological sample or a portion thereof; and (b) subjecting the at least a subset of the plurality of partitions to inductive heating. Subjecting the at least a subset of the plurality of partitions to inductive heating can be at a frequency of at least about 1 cycle per second (Hz), 100 Hz, 500 Hz, 1,000 Hz, 2,000 Hz, 3,000 Hz, 4,000 Hz, 10,000 Hz, or 20,000 Hz. The frequency may be from about 1 Hz to 10,000 Hz, or 1 Hz to 5,000 Hz, or 1 Hz to 1,000 Hz. The frequency of heating can be adjusted by the controller by varying the frequency of energy (e.g., electrical energy) that is provided for inductive heating. In some cases, the plurality of partitions is contained in a vessel and/or the inductive heating is achieved with the aid of a magnetic field, such as an alternating magnetic field. The magnetic field can be provided by an electromagnet.

In some cases, (a) further comprises bringing an aqueous phase in contact with a continuous phase to generate an emulsion comprising aqueous droplets dispersed in the continuous phase. In some cases, the aqueous and continuous phases can be brought into contact at an intersection or junction of a first channel, second channel and third channel whereby the first channel provides the aqueous phase to the junction and the second channel provides the continuous phase to the junction. Due to the immiscibility of the aqueous phase in the continuous phase, aqueous droplets are generated in the continuous phase at the junction and can flow from the junction through the third channel. In some cases, the aqueous and continuous phases can be brought into contact by alternately opening and closing a port or channel that provides discontinuous aliquots of the aqueous phase to a bulk continuous phase.

Examples of such droplet generation include the following. In an aspect, the present disclosure provides an apparatus for generating at least one droplet comprising a biological sample for use in a chemical or biological reaction. The apparatus may comprise a first chamber comprising a first fluid volume and at least one first fluid flow port that is in fluid communication with the first fluid volume. The first fluid volume may retain an aqueous solution comprising the biological sample for use in the chemical or biological reaction. The apparatus may comprise a second chamber comprising a second fluid volume and at least one second fluid flow port that is in fluid communication with the second fluid volume, the second chamber may at least partially circumscribe the first chamber. In some embodiments, the second chamber fully circumscribes the first chamber. The second fluid volume may retain a continuous fluid that is immiscible with the aqueous solution, and the second chamber may be rotatable with respect to the first chamber, or vice versa. In some embodiments, the second chamber is rotatable with respect to the first chamber. In some embodiments, the first chamber is rotatable with respect to the second chamber. The first chamber and/or the second chamber may be cylindrical.

During use, rotation of the first chamber or the second chamber may bring the first fluid flow port in alignment with the second fluid flow port to subject the aqueous solution comprising the biological sample to flow from the first fluid volume to the second fluid volume to generate the at least one droplet upon contact with the continuous fluid, and the at least one droplet may comprise the biological sample or a portion thereof. The first fluid flow port may be in selective alignment with the second fluid flow port upon rotation of the first chamber or the second chamber. The at least one droplet may comprise a plurality of droplets, and each of the plurality of droplets may comprise the biological sample or a portion thereof.

In another aspect, the present disclosure provides a method for generating at least one droplet comprising a biological sample for use in a chemical or biological reaction. The method may comprise: (a) activating an apparatus comprising (1) a first chamber comprising a first fluid volume and at least one first fluid flow port that is in fluid communication with the first fluid volume, and (2) a second chamber comprising a second fluid volume and at least one second fluid flow port that is in fluid communication with the second fluid volume, the first fluid volume may comprise an aqueous solution comprising the biological sample for use in the chemical or biological reaction, and the second chamber may at least partially circumscribe the first chamber. In some embodiments, the second chamber fully circumscribes the first chamber. The second fluid volume may retain a continuous fluid that is immiscible with the aqueous solution, and the second chamber may be rotatable with respect to the first chamber, or vice versa. The method may further comprise (b) rotating the first chamber or the second chamber to bring the first fluid flow port in alignment with the second fluid flow port to subject the aqueous solution comprising the biological sample to flow from the first fluid volume to the second fluid volume to generate the at least one droplet upon contact with the continuous fluid. The rotating in (b) may comprise rotating the second chamber with respect to the first chamber, or rotating the first chamber with respect to the second chamber. The at least one droplet may comprise the biological sample or a portion thereof. The activating may comprise depositing the aqueous solution comprising the biological sample in the first fluid volume.

The at least one droplet may comprise a plurality of droplets, and each of the plurality of droplets may comprise the biological sample or a portion thereof.

The at least one droplet may have a size that is at least partially dependent on a rate of rotation of the first chamber or the second chamber.

The rate of droplet formation may be at least partially dependent on a rate of rotation of the first chamber or the second chamber. For example, when the rate of rotation of the first chamber or the second chamber is high, the rate of droplet formation may be high, and when the rate of rotation of the first chamber or the second chamber is low, the rate of droplet formation may be low. In another example, when the rate of rotation of the first chamber or the second chamber is high, the droplet may have a smaller size, and when the rate of rotation of the first chamber or the second chamber is low, the droplet may have a bigger size.

A droplet of the present disclosure may be an isolated portion of a first fluid (e.g., an aqueous solution) that is completely surrounded by a second fluid (e.g., a continuous fluid). A droplet may be of any suitable shape and it may not necessarily be spherical. The diameter of a droplet, in a non-spherical droplet, is the diameter of a perfect mathematical sphere having the same volume as the non-spherical droplet.

A droplet of the present disclosure may be formed when a portion of a first fluid (e.g., an aqueous fluid) is substantially surrounded by a second fluid (e.g., a continuous fluid). As used herein, a portion of a first fluid is “surrounded” by a second fluid when a closed loop may be drawn around the first fluid through only the second fluid. A portion of a first fluid is “completely surrounded” by a second fluid if closed loops going through only the second fluid may be drawn around the first fluid regardless of direction. A portion of a first fluid is “substantially surrounded” by a second fluid if the loops going through only the second fluid may be drawn around the droplet depending on the direction.

An average size of the droplet may depend on the properties (e.g. flow rate, viscosity) of one or more of the fluids, the size, configuration, or geometry of the chambers, and/or the size, configuration, or geometry of the fluid flow ports.

In some cases, the method further comprises (c) subjecting the at least a subset of the plurality of partitions to cooling. Subjecting the solution to cooling can occur upon decoupling any heating elements from a magnetic field and/or by positioning the at least a subset of the plurality of partitions in a cooling zone comprising one or more cooling units (e.g., a wind tunnel, a convective cooling unit, a cooling block, a thermoelectric device, etc.) that are in thermal communication with the solution. Subjecting the at least a subset of the plurality of partitions to cooling can be achieved via any suitable route of energy transfer, including convective and conductive cooling routes. In some cases, the method further comprises repeating (b) and (c), thereby subjecting the at least a subset of the plurality of partitions to repeated heating and cooling (e.g., thermal cycling). As noted elsewhere herein, thermal cycling can be useful for a host of sample processing and/or biological/chemical reactions, including preparation of the biological sample for a nucleic acid amplification reaction and conducting the nucleic acid amplification reaction.

In some cases, the at least a subset of the plurality of partitions comprises components necessary for conducting a chemical or biological reaction on the biological sample. For example, the biological sample may comprise a nucleic acid molecule and the at least a subset of the plurality of partitions may comprise the biological sample and components necessary for a nucleic acid amplification reaction, with examples of nucleic acid amplification reactions and necessary components for nucleic acid amplification provided elsewhere herein. Where the at least a subset of the plurality of partitions comprises components necessary for conducting a chemical or biological reaction on the biological sample, the method may further comprise conducting the chemical or biological reaction on the biological sample (e.g., conducting nucleic acid amplification reaction(s) in the at least a subset of the plurality of partitions, with or without the aid of thermal cycling). Moreover, the method may also comprise detecting one or more signals indicative of the chemical or biological reaction. Any suitable detector and detection modality can be used, including examples of such provided elsewhere herein.

In another aspect, the disclosure provides a system for processing a biological sample. The system includes a plurality of partitions that comprises the biological sample or a portion thereof and an inductive heating unit that inductively heats the at least a subset of the plurality of partitions. The system also includes a controller operatively coupled to the inductive heating unit. The controller is programmed to direct the inductive heating unit to inductively heat the at least a subset of the plurality of partitions. In some cases, the controller is programmed to direct the inductive heating unit to inductively heat the at least a subset of the plurality of partitions at a frequency of at least about 1 Hz, 100 Hz, 500 Hz, 10,00 Hz, 2,000 Hz, 3,000 Hz, 4,000 Hz, 10,000 Hz, or 20,000 Hz. The frequency may be from about 1 Hz to 10,000 Hz, or 1 Hz to 5,000 Hz, or 1 Hz to 1,000 Hz. The frequency of heating can be adjusted by the controller by varying the frequency of energy (e.g., electrical energy) that it is programmed to provide to the inductive heating unit. In some cases, the system further comprises a vessel that comprises the plurality of partitions. Furthermore, in some cases, the inductive heating unit can comprise an electromagnet that can generate a magnetic field, which may be an alternating magnetic field.

Control of the heating unit by the controller may comprise a feedback-control system wherein the actual temperature of the system, the plurality of partitions, and/or the heating unit is measured, compared against a desired temperature, and the heating unit is instructed by the controller to generate more heat, generate about the same amount of heat, or generate less heat based at least in part on the difference between the desired temperature and the actual temperature. The degree to which heat is generated (e.g., the amount of heat produced by the heating element per unit time, the amount of heat produced, etc.) may at least in part be a function of the actual temperature of the system, the actual temperature of the plurality of partitions, and/or the actual temperature of heating unit at the time of measurement as compared to the desired temperature individually or collectively of the system, the plurality of partitions, and/or the heating unit.

The feedback-control system may, for example, comprise a proportional-integral-derivative controller (PID controller) that seeks to minimize the difference between a desired temperature and the actual temperature; said difference being known as the “error” and said difference charted over time being known as the “error signal.” The PID controller may apply an action in response to the error and/or error signal based on proportional, integral, and/or derivative terms. For example, a PID controller may control the amount of heat generated, the rate at which heat is generated, or the rate at which heat is accumulated by the system or any of its components in response to the difference between a measured temperature and a desired temperature. The response signal (the signal sent by the controller to the elements in response to a difference between a measured temperature and a desired temperature (e.g. a temperature needed for a biological or chemical reaction)) may take any form including but not limited to: a signal that comprises at least in part of a signal scaled to the difference between the measured temperature and the desired temperature (i.e., the error); a signal that comprises at least in part of a signal representing a scaled derivative and/or a scaled partial derivative of the error signal; and/or a signal that comprises at least in part of a signal representing a scaled integration of the error signal over some portion of time.

The feedback-control system may comprise a bang-bang controller (also referred to as an “on-off controller” and a “hysteresis controller”) to control the temperature and/or heat generation of the system. For example, control the heating unit by a controller comprising a bang-bang controller may send an ON or OFF signal such that the heating unit (or at least a subset of one or more heating elements of a heating unit) is either heating (ON) or not (OFF). A non-limiting example of such control includes delivering an ON signal to one or more heating elements of the heating unit when the actual temperature falls below a desired temperature and delivering an OFF signal to one or more heating elements of the heating unit when the actual temperatures falls below a desired temperature. In some embodiments, the controller may deliver an ON signal to one or more heating elements of the heating unit when the actual temperature falls below a threshold temperature and delivering an OFF signal to one or more heating elements of the heating unit when the actual temperatures falls below a threshold temperature. In some embodiments, the controller may modulate the rate at which heat is generated such that the heat flux of one or more heating elements is a function of the duty cycle of the signal of the controller.

The feedback-control system may comprise a pulse-width modulation (PWM) controller wherein the signal sent to one or more heating elements to initiate heating may have at least a portion of its duty cycle and/or signal width over a period of time regulated by the PWM controller. PWM has the benefit of allow the controller to send a certain type of signal (say, for example, a square-waved voltage) to one or more heating elements and to modify the control of the one or more heating elements by modulating at least one width of the signal (for example, the wavelength of the square wave). In some embodiments, the controller may modulate the rate at which heat is generated such that the heat flux of one or more heating elements is a function of the duty cycle of the signal of the controller. A controller utilizing PWM may modulate the width of any signal with a frequency of at least 1 hertz (Hz), 2 Hz, 3 Hz, 4 Hz, 5 Hz, 6 Hz, 7 Hz, 8 Hz, 9 Hz, 10 Hz, 20 Hz, 30 Hz, 40 Hz, 50 Hz, 60 Hz, 70 Hz, 80 Hz, 90 Hz, 100 Hz, 200 Hz, 300 Hz, 400 Hz, 500 Hz, 600 Hz, 700 Hz, 800 Hz, 900 Hz, 1 kilohertz (kHz), 2 kHz, 3 kHz, 4 kHz, 5 kHz, 6 kHz, 7 kHz, 8 kHz, 9 kHz, 10 kHz, 20 kHz, 30 kHz, 40 kHz, 50 kHz, 60 kHz, 70 kHz, 80 kHz, 90 kHz, 100 kHz, 200 kHz, 300 kHz, 400 kHz, 500 kHz, 600 kHz, 700 kHz, 800 kHz, 900 kHz, 1 megahertz (MHz), 2 MHz, 3 MHz, 4 MHz, 5 MHz, 6 MHz, 7 MHz, 8 MHz, 9 MHz, 10 MHz, 20 MHz, 30 MHz, 40 MHz, 50 MHz, 60 MHz, 70 MHz, 80 MHz, 90 MHz, 100 MHz, 200 MHz, 300 MHz, 400 MHz, 500 MHz, 600 MHz, 700 MHz, 800 MHz, 900 MHz, or 1 gigahertz (GHz), or any value in between.

The heating unit of any embodiment may be set to a first temperature and held constant or nearly constant while that which is being heated is being heated. The heating unit of any embodiment may be set to the first temperature and held constant or nearly constant while that which is being heated is heated to the first temperature. Once that which is being heated is heated to the first temperature the heating unit may remain heating at the same first temperature or may transition to a second temperature, different than the first. The second temperature may be less than or greater than the first temperature. For example, the heating unit may be heated to a first temperature of 95° C. while that which is to be heated (e.g., a solution comprising a biological sample) has an initial temperature of 55° C. That which is to be heated may then transition from its initial temperature to the first temperature of the heating unit, in this case rising from 55° C. to 95° C. The transition from an initial temperature to the temperature set by the heating unit may take any amount of time. For example, the time it takes to transition from an initial temperature to the temperature set by the heating unit may take 0.001 seconds (s), 0.01 s, 0.1 s, 1 s, 10 s, or 100 s, or the time it takes to transition from an initial temperature to the temperature set by the heating unit may take on any value in between any two of the aforementioned values.

The temperature of that which is to be heated during any of the aforementioned heating regimes may be below, about equal to, or above the desired temperature at any given time. The heating regime may be underdamped such that the temperature of that which is to be heated overshoots the desired temperature and then oscillates for a period of time until settling to a temperature approximately equal to the desired temperature (e.g., that set by the heating unit, that of the unit, etc.). The heating regime may be critically damped such that the temperature of that which is to be heated achieves the desired temperature level as quickly as possible without overshooting the desired temperature. The heating regime may be overdamped such that the temperature of that which is to be heated achieves the desired temperature level at a rate below that of the critically damped case without overshooting the desired temperature. The heating regime may comprise any combination of aforementioned heating regimes.

The control scheme of any aforementioned control system may be determined at least partially in advance (e.g., choosing to use PWM control, the rate of modulation therein, etc.) and/or at least partially during the time the controller controls the heating unit (e.g., the extent to which the duty cycle is modified by measured conditions). In some embodiments one or more aspects of the control scheme of the controller is known in advance. For example, the extent of PWM required for the heating unit to reach a subset of temperatures (e.g., a first temperature, a second temperature, a third temperature, etc.) may be programmed into the controller prior to heating. In some embodiments, the controller utilizes both offline and online methods of control such as by incorporating previously empirically derived control parameters (an offline method) and modulating heating unit behavior in real time (an online method). The controller of any embodiment may comprise any combination of controllers, control regimes, control parameters, and/or feedback-control systems described herein.

The system may further comprise a first source of an aqueous phase; a second source of a continuous phase; and an emulsion generation unit. The emulsion generation unit is in fluid communication with the first source and the second source and brings the aqueous phase in contact with the continuous phase to generate the emulsion. In some cases, the emulsion generation unit comprises an intersection of a first channel that is in fluid communication with the first source and second channel is in fluid communication with the second source. The intersection may also be in fluid communication with a third channel through which droplets generated at the intersection are transported from the intersection.

For example, the first source and second source may be first and second reservoirs comprising the aqueous phase and continuous phase, respectively, in fluid communication with the intersection via the first and second channels, respectively. With the aid of forced flow, the aqueous phase flows from the first reservoir through the first channel and into the intersection, while the continuous phase flows from the second reservoir through the second channel and into the intersection. At the intersection, an emulsion comprising droplets is generated due to the immiscibility of the aqueous and continuous phases. The generated droplets then flow from the intersection to a third channel for downstream processing. Alternatively, the emulsion generation unit may comprise a port that is alternately openable and closable and in fluid communication with a bulk continuous phase. Upon opening and closing of the port, discontinuous aliquots of aqueous phase flow into the bulk continuous phase to generate the emulsion.

Moreover, the system can further comprise a cooling zone that subjects the at least a subset of the plurality of partitions. A cooling unit (e.g., a wind tunnel, a convective cooling unit, a cooling block, an evaporative cooling unit, a Peltier device, a cooling bath unit) may provide cooling to the cooling zone. Moreover, the controller may comprise one or more computer processors that are individually or collectively programmed to alternately and sequentially position the solution in the magnetic field and in a heating zone in thermal communication with the inductive heating unit that heats the at least a subset of the plurality of partitions with heat generated by the inductive heating unit; and the cooling zone. For example, the at least a subset of the plurality of partitions may be housed in a vessel that is rotatable with respect to the heating (and, perhaps, with respect to the inductive heating unit) and/or cooling zones, or vice versa. The controller may generate signals that actuate rotation of the vessel, a heating zone and/or a cooling zone about an axis of rotation. The heating zone and cooling zone may be angularly separated such that rotation of the vessel about an axis of rotation passes the at least a subset of the plurality of partitions from one of the heating zone and the cooling zone to the other of the two. In some cases, rotation of the magnetic field application unit and/or the cooling zone about an axis of rotation exposes the at least a subset of the plurality of partitions to cyclical heating (and cooling where applicable).

In some cases, the system further comprises a detector. The detector may be positioned adjacent to a vessel comprising the at least a subset of the plurality of partitions or may be positioned at a distance from said vessel. The detector can detect, during detection, one or more signals from the at least a subset of the plurality of partitions that are indicative of a chemical or biological reaction on the biological sample. Any suitable detector and detection modality may be used, including examples of detectors and detection modalities described elsewhere herein.

Various aspects include or provide a plurality of partitions. The plurality of partitions may comprise any suitable type(s) of partitions, with non-limiting examples of partitions that include a droplet (e.g., droplets in an emulsion as described elsewhere herein), a well, a microwell, a hole, a continuous phase of an emulsion, a tube, a spot, a capsule, a bead or any combination thereof. In some cases, a partition may comprise one or more additional partitions.

Additionally, the volume of a partition in a plurality of partitions may have any suitable volume. In some cases, a plurality of partitions may be contained in a volume of at least about 0.001 mL, at least about 0.005 mL, at least about 0.01 mL, at least about 0.05 mL, at least about 0.1 mL, at least about 0.5 mL, at least about 1 mL, at least about 2 mL, at least about 3 mL, at least about 4 mL, at least about 5 mL, at least about 6 mL, at least about 7 mL, at least about 8 mL, at least about 9 mL, at least about 10 mL or more. In some cases, a plurality of partitions may be contained in a volume of at most about 10 mL, at most about 9 mL, at most about 8 mL, at most about 7 mL, at most about 6 mL, at most about 5 mL, at most about 4 mL, at most about 3 mL, at most about 2 mL, at most about 1 mL, at most about 0.5 mL, at most about 0.1 mL, at most about 0.05 mL, at most about 0.01 mL, at most about 0.005 mL, at most about 0.001 mL.

Additionally, a plurality of partitions can be contained in any suitable volume. In some cases, the plurality of partitions may be contained in a volume that is less than or equal to about 50 milliliters (mL), 40 mL, 30 mL, 20 mL, 10 mL, 5 mL, 1 mL, 100 microliters (uL), 10 uL, 1 uL, 500 nanoliters (nL), 100 nL, or 10 nL. Each of the partitions may have a volume in the picoliter (pL) or nanoliter (nL) range up to the microliter (uL) range. The volume of a partition may be at least about 1 pL, 10 pL, 100 pL, 500 pL, 1 nL, 100 nL, 500 nL, 1 uL, 100 uL, 1,000 uL, or greater. In some cases, a partition has a volume that is less than or equal to about 1,000 uL, 100 uL, 50 uL, 40 uL, 30 uL, 20 uL, 10 uL, 1 uL, 500 nL, 100 nL, or 1 nL.

A plurality of partitions may comprise any suitable number of partitions. In some cases, a relatively high number of partitions may be useful for certain downstream analysis. For example, in the case of digital PCR, a high number of partitions can improve assay results and also ensure that, on average, a plurality of partitions comprises less than 1 template nucleic acid molecule per partition. In some cases, a plurality of partitions may comprise at least about 2 partitions, at least about 10 partitions, at least about 50 partitions, at least about 100 partitions, at least about 500 partitions, at least about 1,000 partitions, at least about 2,500 partitions, at least about 5,000 partitions, at least about 7,500 partitions, at least about 10,000 partitions, at least about 25,000 partitions, at least about 50,000 partitions, at least about 75,000 partitions, at least about 100,000 partitions, at least about 200,000 partitions, at least about 300,000 partitions, at least about 400,000 partitions, at least about 500,000 partitions, at least about 750,000 partitions, at least about 1,000,000 partitions, at least about 10,000,000 partitions, at least about 100,000,000 partitions, at least about 1,000,000,000 partitions or more.

In various aspects, a solution and/or vessel comprising at least a subset of a plurality of partitions may also comprise one or more heating elements that generate heat upon inductive coupling to a magnetic field. Where a solution comprises the one or more heating elements, at least a subset of the one or more heating elements can be dissolved, suspended or floating in the solution. In some cases, at least a given one of the one or more heating elements are in a given one of a plurality of partitions. The one or more heating elements may be in separate partitions from those containing a biological sample (e.g., in heating droplets 303 of a population of droplets also comprising separate reaction droplets 302 having a biological sample as shown in FIG. 3A and described elsewhere herein) or may be present in the same partitions as a biological sample (e.g., in reaction droplets 312 of a population of droplets as shown in FIG. 3B and described elsewhere herein). Moreover, in various aspects, at least a subset of a plurality of partitions can comprise components necessary for conducting a chemical or biological reaction on a biological sample. In some cases, the chemical or biological reaction is a nucleic acid amplification reaction.

In various aspects, methods and systems for processing a biological sample can include heating of a solution or population of partitions or heat a solution or a population of partitions at relatively high temperature ramp rates. Relatively high temperature ramp rates can be advantageous for a number of reasons, including reduced sample processing time and reduced time of exposure of a biological sample (and any additional materials) to elevated temperatures. For example, a system can heat, or a method can include heating a solution or population of partitions, at a rate of at least about 5° C./second (“s”), at least about 10° C./s, at least about 15° C./s, at least about 20° C./s, at least about 25° C./s, at least about 30° C./s, at least about 35° C./s, at least about 40° C./s, at least about 45° C./s, at least about 50° C./s, at least about 55° C./s, at least about 60° C./s, at least about 65° C./s, at least about 70° C./s, at least about 75° C./s, at least about 80° C./s, at least about 85° C./s, at least about 90° C./s, at least about 95° C./s, at least about 100° C./s, at least about 105° C./s, at least about 110° C./s, at least about 115° C./s, at least about 120° C./s, at least about 150° C./s, at least about 200° C./s, or more. Once heating is terminated, the solution or population of partitions may cool at a cooling rate of at least about 5° C./ s, at least about 10° C./s, at least about 15° C./s, at least about 20° C./s, at least about 25° C./s, at least about 30° C./s, at least about 35° C./s, at least about 40° C./s, at least about 45° C./s, at least about 50° C./s, at least about 55° C./s, at least about 60° C./s, at least about 65° C./s, at least about 70° C./s, at least about 75° C./s, at least about 80° C./s, at least about 85° C./s, at least about 90° C./s, at least about 95° C./s, at least about 100° C./s, at least about 105° C./s, at least about 110° C./s, at least about 115° C./s, at least about 120° C./s, at least about 150° C./s or more.

In various aspects, methods and systems for processing a biological sample described herein may provide for localized heating and/or cooling. Such localized heating and/or cooling may be more efficient than bulk heating and/or cooling. In some examples, heating is implemented inductively to generate localized heating of partitions (e.g., droplets) and/or in some cases a solution surrounding the partitions. As described elsewhere herein, heating can be effected with aid of one or more heating elements. The positioning of heating elements within partitions and/or within a solution comprising components to-be-heated provides heat in much closer proximity to the species subject to heating. As less heat is lost to the surrounding environment with localized heating, less energy (in some cases, substantially less energy) is used for heating and more rapid heating can be achieved when compared to bulk heating at equivalent energy input.

Once heating is terminated, rapid cooling, may ensue, in some cases due to the surrounding environment being much cooler than the species (e.g., partitions, solution) being heated. As is discussed above, localized heating results in less energy needed for heating. As less energy is supplied for heating, energy transfer amounts are also lower for cooling. The relatively low temperature of a surrounding environment compared to the temperature of localized heating regions (e.g., a solution, within a population of partitions, within a partition) can rapidly transfer energy from the localized heating regions. For example, heating elements can be contained within droplets in an emulsion, such that heating is localized to the interior of the droplets. Conversely, relatively low energy is transferred to the continuous phase of the emulsion, such that the continuous phase remains at substantially the same temperature. Upon termination of heating, the large temperature gradient between the droplet interiors and the continuous phase of the emulsion can drive rapid cooling in the droplet interiors. Moreover, such cooling can also avoid inefficiencies (and, thus, slower cooling rates) associated with bulk cooling, such as inefficiencies associated with cooling bulk species that are not subject to heating.

Methods and systems of the present disclosure may be used for localized heating. In localized heating, a relatively local volume may be heated at a higher rate than a larger surrounding volume. As an alternative or in addition to, methods and systems provided herein may be used to perform bulk (e.g., 1 milliliter to 5 milliliter volume) heating. In bulk heating, an entirely of a given volume may be heated.

In various aspects, methods and system for processing a biological sample described herein can be useful for fast thermal cycling, whereby a solution of population of partitions is repeatedly heated and cooled. For example, during a nucleic acid amplification reaction, thermal cycling may repeatedly cycle the temperature of the solution or population of partitions through a denaturation temperature (e.g., in the range of 80° C.-100° C., whereby double-stranded nucleic acid separates into its single strands) and an elongation temperature (e.g., in the range of 30° C.-80° C., whereby nucleotides are incorporated into a template nucleic acid). Relatively high temperature thermal cycle times can be advantageous for a number of reasons, including reduced sample processing time. For example, a system can complete a single thermal cycle or a method can include completion of a single thermal cycle of a solution in at most about 5 minutes (“min”), at most about 4 min, at most about 3 min, at most about 2 min, at most about 1min, at most about 45 seconds (“s”), at most about 30 s, at most about 25 s, at most about 20 s, at most about 15 s, at most about 10 s, at most about 9 s, at most about 8 s, at most about 7 s, at most about 6 s, at most about 7 s, at most about 6 s, at most about 5 s, at most about 4 s, at most about 3 s, at most about 2 s, at most about 1 s, at most about 0.5 s, at most about 0.1 s or less.

Methods and systems of the present disclosure may be used to subject a sample to one or more cycles of heating and cooling, such as at least 1, 2, 3, 4, 5, 6, 7, 8, 9, 10, 20, 30, 40, 50, 60, 70 80, 90, or 100 cycles of heating and cooling. Heating and cooling may be performed by incubating the sample at a denaturing temperature for a denaturation duration and incubating the sample at an elongation temperature at an elongation duration.

Denaturation temperatures may vary depending upon, for example, the particular biological sample analyzed, the particular source of target nucleic acid (e.g., viral particle, bacteria) in the biological sample, the reagents used, and/or the desired reaction conditions. For example, a denaturation temperature may be from about 80° C. to about 110° C. In some examples, a denaturation temperature may be from about 90° C. to about 100° C. In some examples, a denaturation temperature may be from about 90° C. to about 97° C. In some examples, a denaturation temperature may be from about 92° C. to about 95° C. In still other examples, a denaturation temperature may be about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

Denaturation durations may vary depending upon, for example, the particular biological sample analyzed, the particular source of target nucleic acid (e.g., viral particle, bacteria) in the biological sample, the reagents used, and/or the desired reaction conditions. For example, a denaturation duration may be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, a denaturation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

Elongation temperatures may vary depending upon, for example, the particular biological sample analyzed, the particular source of target nucleic acid (e.g., viral particle, bacteria) in the biological sample, the reagents used, and/or the desired reaction conditions. For example, an elongation temperature may be from about 30° C. to about 80° C. In some examples, an elongation temperature may be from about 35° C. to about 72° C. In some examples, an elongation temperature may be from about 45° C. to about 65° C. In some examples, an elongation temperature may be from about 35° C. to about 65° C. In some examples, an elongation temperature may be from about 40° C. to about 60° C. In some examples, an elongation temperature may be from about 50° C. to about 60° C. In still other examples, an elongation temperature may be about 35°, 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

Elongation durations may vary depending upon, for example, the particular biological sample analyzed, the particular source of target nucleic acid (e.g., viral particle, bacteria) in the biological sample, the reagents used, and/or the desired reaction conditions. For example, an elongation duration may be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, an elongation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) necessary to obtain a detectable amplified product (e.g., a detectable amount of amplified DNA product that is indicative of the presence of a target RNA in a biological sample). For example, the number of cycles necessary to obtain a detectable amplified product (e.g., a detectable amount of DNA product that is indicative of the presence of a target RNA in a biological sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target RNA in a biological sample) may be obtained at a cycle threshold value (Ct) of less than 100, 75, 70, 65, 60, 55, 50, 45, 40, 35, 30, 25, 20, 15, 10, or 5.

The time for which amplification yields a detectable amount of amplified product indicative of the presence of a target nucleic acid amplified can vary depending upon the biological sample from which the target nucleic acid was obtained, the particular nucleic acid amplification reactions to be conducted, and the particular number of cycles of amplification reaction desired. For example, amplification of a target nucleic acid may yield a detectable amount of amplified product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a target RNA may yield a detectable amount of amplified DNA product indicative to the presence of the target RNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, a reaction mixture may be subjected to a plurality of series of primer extension reactions. An individual series of the plurality may comprise multiple cycles of a particular primer extension reaction, characterized, for example, by particular denaturation and elongation conditions as described elsewhere herein. Generally, each individual series differs from at least one other individual series in the plurality with respect to, for example, a denaturation condition and/or elongation condition. An individual series may differ from another individual series in a plurality of series, for example, with respect to any one, two, three, or all four of denaturing temperature, denaturing duration, elongation temperature, and elongation duration. Moreover, a plurality of series may comprise any number of individual series such as, for example, at least about or about 2, 3, 4, 5, 6, 7, 8, 9, 10, or more individual series.

For example, a plurality of series of primer extension reactions may comprise a first series and a second series. The first series, for example, may comprise more than ten cycles of a primer extension reaction, where each cycle of the first series comprises (i) incubating a reaction mixture at about 92° C. to about 95° C. for no more than 30 seconds followed by (ii) incubating the reaction mixture at about 35° C. to about 65° C. for no more than about one minute. The second series, for example, may comprise more than ten cycles of a primer extension reaction, where each cycle of the second series comprises (i) incubating the reaction mixture at about 92° C. to about 95° C. for no more than 30 seconds followed by (ii) incubating the reaction mixture at about 40° C. to about 60° C. for no more than about 1 minute. In this particular example, the first and second series differ in their elongation temperature condition. The example, however, is not meant to be limiting as any combination of different elongation and denaturing conditions may be used.

In some embodiments, the ramping time (i.e., the time the thermal cycler takes to transition from one temperature to another) and/or ramping rate can be important factors in amplification. For example, the temperature and time for which amplification yields a detectable amount of amplified product indicative of the presence of a target nucleic acid can vary depending upon the ramping rate and/or ramping time. The ramping rate can impact the temperature(s) and time(s) used for amplification.

In some cases, the ramping time and/or ramping rate can be different between cycles. In some situations, however, the ramping time and/or ramping rate between cycles can be the same. The ramping time and/or ramping rate can be adjusted based on the sample(s) that are being processed.

In some situations, the ramping time between different temperatures can be determined, for example, based on the nature of the sample and the reaction conditions. The exact temperature and incubation time can also be determined based on the nature of the sample and the reaction conditions. In some embodiments, a single sample can be processed (e.g., subjected to amplification conditions) multiple times using multiple thermal cycles, with each thermal cycle differing for example by the ramping time, temperature, and/or incubation time. The best or optimum thermal cycle can then be chosen for that particular sample. This provides a robust and efficient method of tailoring the thermal cycles to the specific sample or combination of samples being tested.

In some embodiments, a target nucleic acid may be subjected to a denaturing condition prior to initiation of a primer extension reaction. In the case of a plurality of series of primer extension reactions, the target nucleic acid may be subjected to a denaturing condition prior to executing the plurality of series or may be subjected to a denaturing condition between series of the plurality. For example, the target nucleic acid may be subjected to a denaturing condition between a first series and a second series of a plurality of series. Non-limiting examples of such denaturing conditions include a denaturing temperature profile (e.g., one or more denaturing temperatures) and a denaturing agent.

An advantage of conducting a plurality of series of primer extension reaction may be that, when compared to a single series of primer extension reactions under comparable denaturing and elongation conditions, the plurality of series approach yields a detectable amount of amplified product that is indicative of the presence of a target nucleic acid in a biological sample with a lower cycle threshold value. Use of a plurality of series of primer extension reactions may reduce such cycle threshold values by at least about or about 1%, 5%, 10%, 15%, 20%, 25%, 30%, 35%, 40%, 45%, 50%, 55%, 60%, 65%, 70%, 75%, 80%, 85%, 90%, or 95% when compared to a single series under comparable denaturing and elongation conditions.

In some embodiments, a biological sample may be preheated prior to conducting a primer extension reaction. The temperature (e.g., a preheating temperature) at which and duration (e.g., a preheating duration) for which a biological sample is preheated may vary depending upon, for example, the particular biological sample being analyzed. In some examples, a biological sample may be preheated for no more than about 60 minutes, 50 minutes, 40 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, 45 seconds, 30 seconds, 20 seconds, 15 seconds, 10 seconds, or 5 seconds. In some examples, a biological sample may be preheated at a temperature from about 80° C. to about 110° C. In some examples, a biological sample may be preheated at a temperature from about 90° C. to about 100° C. In some examples, a biological sample may be preheated at a temperature from about 90° C. to about 97° C. In some examples, a biological sample may be preheated at a temperature from about 92° C. to about 95° C. In still other examples, a biological sample may be preheated at a temperature of about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

Various aspects include a detector that detects a signal indicative of a chemical or biological reaction on a biological sample or detecting such signals. In some cases, the signals are electronic signals generated be a detector. Moreover, a chemical or biological reaction may be detected via the detection of a product (e.g., directly detecting the product itself, detecting a species indicative of the formation of product such as a reporter agent) or via one or more of its reactants (e.g., detecting the disappearance of a reactant, including the biological sample, detecting a species indicative of the disappearance of a reactant, etc.). Any suitable detector and associated detection modality can be used for detection. The particular type of detector and/or detection used may depend, for example, on the particular chemical or biological reaction, the type of any vessel in which a chemical or biological reaction takes places, whether or not a reporter agent is used, and, if a reporter agent was is used, the particular type of reporter agent. Non-limiting examples of detection methods include optical detection, spectroscopic detection, electrostatic detection, electrochemical detection, and the like. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel based techniques, such as, for example, gel electrophoresis. Electrochemical detection methods include, but are not limited to, electrochemical detection of appropriate species after high-performance liquid chromatography separation of the species. Appropriate detectors are available for each of the example detection methods described herein, with examples that include a spectrophotometer, an imaging device (e.g., microscopes, cameras, etc.), an electrospray detector, a time-of-flight detector, an NMR detector, a conductivity detector or any combination thereof.

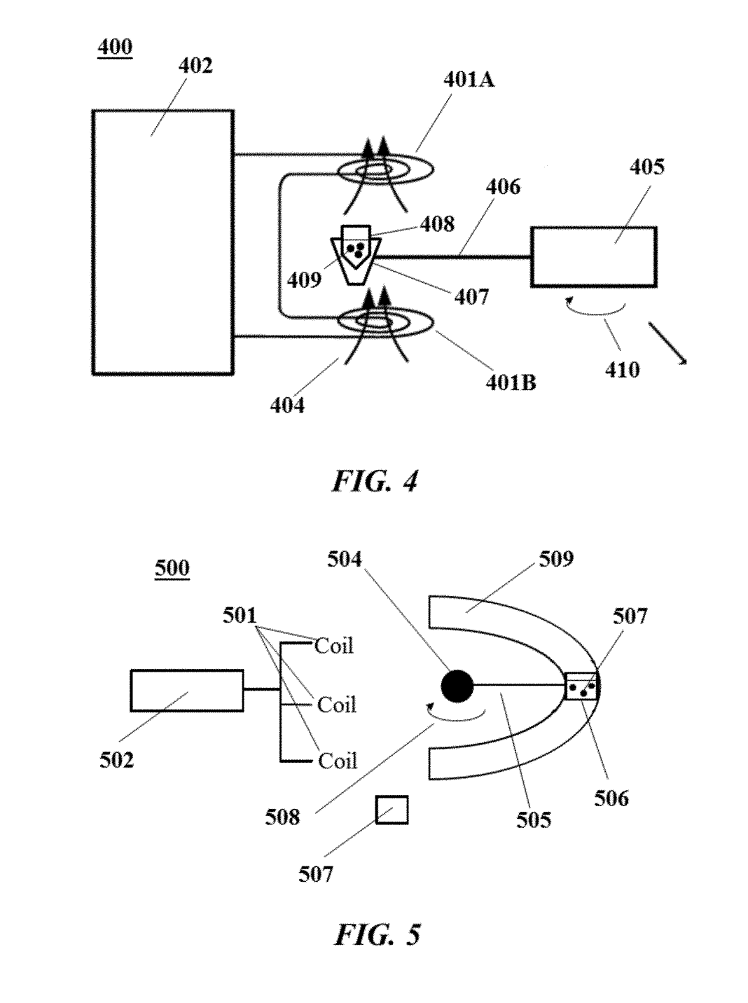

An example system and an associated method for biological processing are schematically depicted in FIG. 4. As is shown in FIG. 4, the system 400 includes an inductive heating unit that comprises an electromagnetic comprising two coils 401A and 401B. Coils 401A and 401B may be structurally similar or identical. Moreover, although not shown, at least a portion of coils 401A and/or 401B may be shielded to minimize electrical interference from the surrounding environment. The coils 401A and 401B are in electrical communication with circuitry 402 that supplies electrical current to the coils 401A and 401B. The circuitry 402 can be in electrical communication with a controller that is programmed to direct the circuitry and/or associated electrical components to provide electrical current through the circuitry 402 and to the coils 401A and 401B. Upon providing electrical current through the coils 401A and 401B, a magnetic field 404 is generated, including in the region between the coils 401A and 401B.

Where alternating current is provided to the coils 401A and 401B, an alternating magnetic field is generated. While only one set of coils is shown in FIG. 4, the system 400 can include any suitable number of coils that may provide multiple regions having a magnetic field 404.

The system 400 also includes a rotatable member 405 that is coupled to an arm 406. The rotatable member 405 can be a piece that is coupled to a component that rotates, such as an electric motor or a manually-powered rotation device. In turn, the arm 406 is coupled to a vessel receiver 407 in which a vessel can be secured in place. A vessel 408 comprising a biological sample and heating elements 409 can be placed in the vessel receiver 407. Alternatively, the vessel 408 may comprise one or more attachment points that couple the vessel directly to the arm 406. While only one arm 406 and rotatable member 405 are shown in FIG. 4, the system 400 can include multiple rotating members 405 and/or each rotating member can comprise multiple arms 406 for processing a plurality of biological samples.

A controller coupled to the system 400 or a part of the system 400 can be programmed to actuate the rotatable member 405 to rotate 410 about an axis of rotation. Upon rotation 410 of the rotatable member 405, the vessel receiver 407 (via arm 406) and, thus, vessel 408 and its contained biological sample also rotate about the axis of rotation. Rotation 410, thus, positions the biological sample in and out of the magnetic field 404 generated by the coils 401A and 401B. While positioned in the magnetic field 404 (e.g., a heating zone), the magnetic field 404 generates eddy currents in the heating elements 409 and, thus, the biological sample is subjected to inductive heating. In some cases, vessel 408 may also comprise a structural component having a heating element that can also provide heating to the biological sample when the vessel 408 is positioned in the magnetic field 404. Once rotated out of the magnetic field 404, eddy currents are no longer generated in the heating elements 409 and the sample can cool (e.g., via convective air flow as it rotates). While in the magnetic field 404, heating of the biological sample can give rise to relatively fast temperature ramp rates as is described elsewhere herein. Energy losses to the surrounding environment can also be minimized due to quick heating times and quick cooling.

Repeated rotation of the biological sample in and out of the magnetic field (and, thus, repeated heating and cooling) effectively thermal cycles the biological sample. As noted elsewhere herein, thermal cycling can be useful for a host of chemical and biological reactions, including nucleic acid amplification reactions. Although not shown in FIG. 4, one or more cooling zones can be angularly separated from the magnetic field, such that when rotated through the sample is cooled. As is noted above, a cooling zone may comprise convective air flow generated by the rotation of the vessel 408. A cooling zone may also cool via an associated cooling unit that is in thermal communication with the cooling zone. Any suitable number of cooling zones may be positioned about the axis of rotation. As is described elsewhere herein, relatively fast thermal cycle times can be achieved.

An example system and an associated method for biological processing are schematically depicted in FIG. 5. As is shown in FIG. 5, the system 500 includes an inductive heating unit that comprises an electromagnetic comprising a plurality of coils 501. Although not shown, at least a portion of one or more of the coils 501 may be shielded to minimize electrical interference from the surrounding environment. The coils 501 are in electrical communication with circuitry 502 that supplies electrical current to the coils 501. The circuitry 502 can be in electrical communication with a controller that is programmed to direct the circuitry and/or associated electrical components to provide electrical current through the circuitry 502 and to the coils 501. Upon providing electrical current through the coils 501, a magnetic field is generated, including in the regions between the coils 501. Where alternating current is provided to the coils 501, an alternating magnetic field is generated. While only three coils are shown in FIG. 5, the system 500 can include any suitable number of coils, including numbers greater than or less than three.

The system 500 also includes a rotatable member 504 that is coupled to an arm 505. The rotatable member 505 can be a piece that is coupled to a component that rotates, such as an electric motor or a manually-powered rotation device. In turn, the arm 505 is coupled to a vessel 506 that comprises a biological sample and heating elements 507. The vessel 506 may comprise one or more attachment points that couple the vessel directly to the arm 505. Alternatively, the vessel 506 can be placed in a vessel receiver (not shown) that is coupled to the arm 505. While only one arm 505 and rotatable member 504 are shown in FIG. 5, the system 500 can include multiple rotating members 504 and/or each rotating member can comprise multiple arms 505 for processing a plurality of biological samples.

Furthermore, the system 500 comprises a detector 507 that can detect signals indicative of a chemical or biological reaction that takes places in the vessel 506. In some cases, the vessel includes one or more reporter agents that are detectable by the detector 507. For example, upon heating (and cooling), a nucleic acid amplification reaction may take place in the vessel 506. A reporter agent that generates a signal indicative of the nucleic acid amplification reaction can be detected by the detector 507.

A controller coupled to the system 500 can be programmed to actuate the rotatable member 504 to rotate 508 about an axis of rotation. Upon rotation 508 of the rotatable member 504, the vessel 507 and its contained biological sample also rotate about the axis of rotation. Rotation 504, thus, positions the biological sample in and out of the magnetic field generated by the coils 501. While positioned in the magnetic field (e.g., a heating zone), the magnetic field generates eddy currents in the heating elements 507 and, thus, the biological sample is subjected to inductive heating. In some cases, vessel 506 may also comprise a structural component having a heating element that can also provide heating to the biological sample when the vessel 507 is positioned in the magnetic field. Once rotated out of the magnetic field, eddy currents are no longer generated in the heating elements 507 and the sample can cool (e.g., via convective air flow as it rotates, including through wind tunnel 509). While in the magnetic field, heating of the biological sample can give rise to relatively fast temperature ramp rates as is described elsewhere herein. Energy losses to the surrounding environment can also be minimized due to quick heating times and quick cooling.

Repeated rotation of the biological sample in and out of the magnetic field (and, thus, repeated heating and cooling) effectively thermal cycles the biological sample. As noted elsewhere herein, thermal cycling can be useful for a host of chemical and biological reactions, including nucleic acid amplification reactions. As shown in FIG. 5, the system 500 includes a wind tunnel 509 that is angularly separated from the inductive heating unit and cools the sample when rotated through the wind tunnel 509. Moreover, while only one wind tunnel 509 is shown, the system can include any suitable number of wind tunnels or additional cooling zones that may be positioned about the axis of rotation. As is described elsewhere herein, relatively fast thermal cycle times can be achieved.

As the vessel 507 rotates out of the wind tunnel 509, the detector 507 can detect a signal indicative of a chemical or biological reaction coming from the vessel. The vessel may comprise a reporter agent that emits a detectable signal. During each rotation, the signal can be measured and the detected signals sent back to a controller for further processing. In some cases, a computer control system as described elsewhere herein can display information (e.g., reaction detection, reaction yield, identification of reaction products, etc.) related to the chemical or biological reaction.

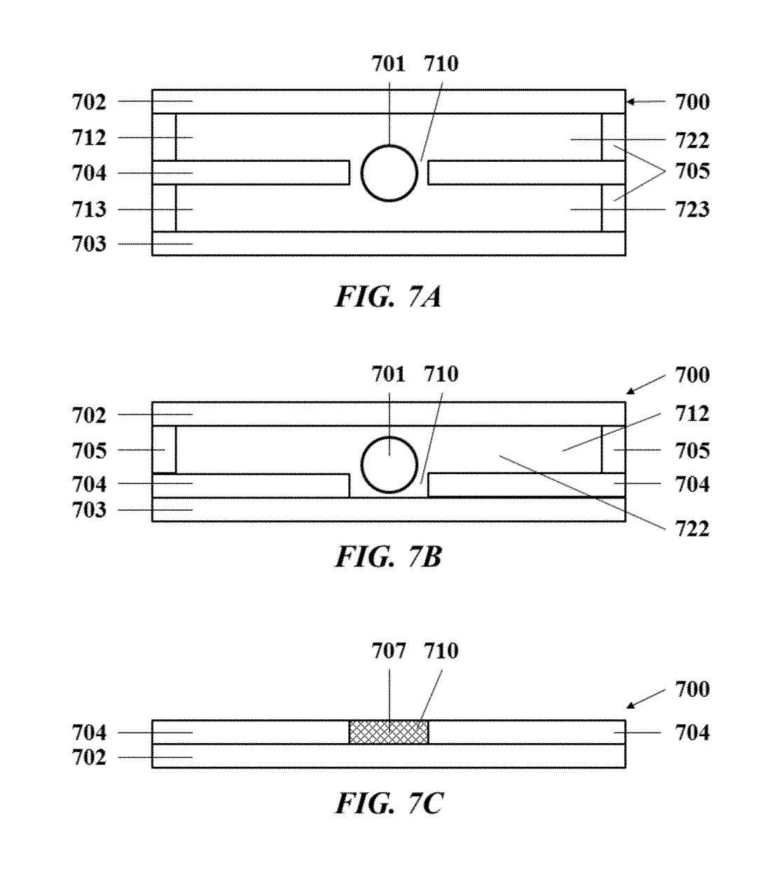

A cross-sectional view of an exemplary support system 700 associated with the methods and systems for biological processing described herein is illustrated in FIGS. 7A-7C. The support system 700 of this or any embodiment may be a portion of a sample processing unit. The sample processing unit (e.g., via the support system 700) may comprise a plurality of wells (e.g., a plurality of supports) and a fluid flow path in fluid communication with the plurality of wells. Flow of a plurality of droplets through the fluid flow path to the plurality of wells such that the plurality of droplets is deposited within the plurality of wells may be controlled via a controller or may be executed manually. Directing the flow of the plurality of droplets may comprise directing the plurality of droplets along a first channel (such as the first channel 722 of FIG. 7A) or a second channel (such as the second channel 723 of FIG. 7A) or both and providing a first liquid phase in the first channel ad a second liquid phase in the second channel to retain the plurality of droplets in the plurality of wells. The first liquid phase of may differ from the second liquid phase, though both can be immiscible with the droplet and/or the plurality of droplets. At least one heating element may be used to convert electrical energy or electromagnetic energy into thermal energy and thereby subject the plurality of droplets to heating. Such heating may at least in part process the biological sample.

The support system 700 may be used to immobilize a sample or a portion of a sample. As illustrated in FIGS. 7A-7B the sample may comprise one or more droplets 701of a solution (e.g., an aqueous solution comprising the biological sample or a portion of the biological sample in an emulsion). The one or more droplets 701 may be of any type of droplet described herein including reaction droplets, heating droplets, or empty droplets.

Turning now to FIG. 7A, the support system 700 may comprise a first bounding layer 702 and a second bounding layer 703 between which the droplet 701 may lie in an opening 710 of a support 704. The first bounding layer 702 and the second bounding layer 703 may individually or collectively comprise an optically clear material such as an optically clear plastic (e.g. acrylic, polycarbonate, etc.), a glass, an organic material, etc. In some embodiments, in additional to optical clarity, the material that comprises the first bounding layer 702 or the second bounding layer 703 may be electrically conductive. Furthermore, the first bounding layer 702 and/or the second bounding layer 703 may comprise a thermoelectric material that generates heat upon activation by either being subjected to a potential or being injected with a current. An example of an optically transparent and electrically conductive material that may comprise the first bounding layer 702 or the second bounding layer 703 or both may be indium tin oxide or other transparent or semi-transparent material that generates thermal energy.

The first bounding layer 702 may at least in part demarcate a first channel 722 within which may reside a first fluid 712 Similarly, the second bounding layer 703 may at least in part demarcate a second channel 723 within which may reside a second fluid 713. In some embodiments, support 704 may at least in part demarcate either the first channel 722 or the second channel 723 or bother. The combination of the first bounding layer 702 and the support 704 may at least in part demarcate the first channel 722 and/or the combination of the second bounding layer 703 and the support 704 may at least in part demarcate the second channel 723.

The first bounding layer 702 or the second bounding layer, or both, may individually or collectively comprise a heating element. The heating element(s) may be of any type described herein (e.g., an inductive heating element, a thermoelectric heating element, etc.). The support system 700 may be heated via the first bounding layer 702, the second bounding layer 703, both, or neither. For those embodiments wherein both the first bounding layer 702 and the second bounding layer 703 comprise a heating element, heat may be generated by both layers simultaneously or sequentially or any combination thereof.

Thermal contact between the solution containing the biological sample (in this illustrated embodiment, the droplet 701) and the first bounding layer 702 may be facilitated by a first fluid 712. Similarly, thermal contact between the solution containing the biological sample (e.g., the droplet 701) and the second bounding layer 703 may be facilitated by a second fluid 713. The first fluid 712 and the second fluid 713 may comprise any fluid described herein, such as an oil. The first fluid 712 and the second fluid 713 may comprise different fluids. The fluids comprising the first fluid 712 and the second fluid 713 may differ in their chemical composition, their viscosity, their density, etc. In those cases in which the densities of the first fluid 712 and the second fluid 713 differ, the first fluid 712 may be less dense than the solution containing the biological sample and the second fluid 713 may be more dense than the solution containing the biological sample so that the solution containing the biological sample may rest between the two fluids, for instance, in the opening 710 of the support 704.

The first bounding layer 702 or the second bounding layer 703 or both may comprise a coating (not illustrated) that electrically insulates the first bounding layer 702 or the second bounding layer 703 or both from other elements (e.g., from the coupling element 705, the first fluid 712, the second fluid 713, etc.). For example, the first bounding layer 702 or the second bounding layer 703 or both may comprise a combination of indium tin oxide and polyethylene terephthalate (PET) (e.g., PET-P, PET-G, etc.). The first bounding layer 702 or the second bounding layer 703 or both may comprise carbon, graphite, plastic, metal (e.g., steel, nickel, aluminum, etc.), or any combination thereof. For example, sheets of carbon may be deposited on, coated on, layer onto, sprayed onto, fused to, bound on, or coupled to the first bounding layer 702, the second bounding layer 703, or any component of the support system 700, or any combination thereof. The first bounding layer 702 or the second bounding layer 703 or both may comprise an electrically non-conductive materials, such as one or more plastics, carbon, graphite, etc. In some embodiments, the first bounding layer 702 or the second bounding layer 703 or any component of the support system 700 may be formed via injection molding.

The first bounding layer 702 or the second bounding layer or both may individually or collectively have a thickness of less than or about 1 micrometers (μm), 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 millimeters (mm), 2 mm, 3 mm, 4 mm, 5 mm, 6 mm, 7 mm, 8 mm, 9 mm, 1 centimeters (cm), 2 cm, 3 cm, 4 cm, 5 cm, 6 cm, 7 cm, 8 cm, 9 cm, 10 cm, or they may take on any value in between. In some embodiments, the first bounding layer 702 or the second bounding layer or both may individually or collectively have a thickness of no more than about 1 micrometers (μm), 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 millimeters (mm), 2 mm, 3 mm, 4 mm, 5 mm, 6 mm, 7 mm, 8 mm, 9 mm, 1 centimeters (cm), 2 cm, 3 cm, 4 cm, 5 cm, 6 cm, 7 cm, 8 cm, 9 cm, 10 cm, or they may take on any value in between Similarly, the support system 700 in some embodiments has an overall thickness of less than or about 1 micrometers (μm), 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 millimeters (mm), 2 mm, 3 mm, 4 mm, 5 mm, 6 mm, 7 mm, 8 mm, 9 mm, 1 centimeters (cm), 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm. In some embodiments, the support system 700 has an overall thickness of no more than about 1 micrometers (μm), 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 millimeters (mm), 2 mm, 3 mm, 4 mm, 5 mm, 6 mm, 7 mm, 8 mm, 9 mm, 1 centimeters (cm), 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm.

The first bounding layer 702 and the second bounding layer 703 may each be coupled to the support 704 via a coupling element 705.

The support 704 may comprise a partition of any type described herein. For example, the support 704 may comprise a material mesh (such as nickel, chromium, stainless steel, etc.). The support may be sized and shaped to hold a suitable volume of material (e.g., of the sample, of the solution comprising the sample, etc.). In some cases, the support may hold a volume of at least about 0.001 mL, at least about 0.005 mL, at least about 0.01 mL, at least about 0.05 mL, at least about 0.1 mL, at least about 0.5 mL, at least about 1 mL, at least about 2 mL, at least about 3 mL, at least about 4 mL, at least about 5 mL, at least about 6 mL, at least about 7 mL, at least about 8 mL, at least about 9 mL, at least about 10 mL or more. In some cases, the support 704 may hold a volume of at most about 10 mL, at most about 9 mL, at most about 8 mL, at most about 7 mL, at most about 6 mL, at most about 5 mL, at most about 4 mL, at most about 3 mL, at most about 2 mL, at most about 1 mL, at most about 0.5 mL, at most about 0.1 mL, at most about 0.05 mL, at most about 0.01 mL, at most about 0.005 mL, at most about 0.001 mL. Moreover, the support 704 may be contained in any suitable volume. In some cases, the support 704 may be circumscribe a volume that is less than or equal to about 50 milliliters (mL), 40 mL, 30 mL, 20 mL, 10 mL, 5 mL, 1 mL, 100 microliters (uL), 10 uL, 1 uL, 500 nanoliters (nL), 100 nL, or 10 nL. The support 704 may circumscribe a volume in the picoliter (pL) or nanoliter (nL) range up to the microliter (uL) range. The volume circumscribed by the support 704 may be at least about 1 pL, 10 pL, 100 pL, 500 pL, 1 nL, 100 nL, 500 nL, 1 uL, 100 uL, 1,000 uL, or greater. In some cases, a support 704 may hold a volume that is less than or equal to about 1,000 uL, 100 uL, 50 uL, 40 uL, 30 uL, 20 uL, 10 uL, 1 uL, 500 nL, 100 nL, or 1 nL.

The support 704 may be configured to retain a plurality of droplets (droplets that may individually or collectively comprise a biological sample or a portion thereof) before, during, or after heating of the plurality of droplets. At least one heating element (of a possible plurality of heating elements) may be in thermal communication with the plurality of wells.

The coupling element 705 may comprise an adhesive, a glue, a tape, a locking mechanism, a weld, a solder joint, or a stitched region.

The opening 710 may be sized and shaped to receive the droplet 701. In some cases the opening 710 may have a cross-sectional diameter that is slight less than the projected diameter of the droplet 701 such that only a portion of the droplet 701 may fit through the opening 710. The droplet may be immobilized via an interference fit, may be held in place by van der Waals reactions, and/or may be directed and/or supported by capillary forces. As the droplet 701 is immobilized in the support system 700, the droplet may not retain its shape. In those instances in which the droplet 701 does not retain its shape while immobilized in the support system 700, the droplet may take on the shape or a portion of the shape of the opening 710. In some cases, the opening 710 may only allow a droplet 701 to enter from one side of the opening 710 (e.g., a first side) while precluding it from exiting from another side of the opening 710 (e.g., a second side). In some cases, the opening 710 may permit unidirectional flow.

The opening 710 may permit fluid communication between the first channel 722 and the second channel 723 such that a first opening in the first channel 722 is in fluid communication with a second opening in the second channel 723.

FIG. 7B illustrates a support system 700 similar to that shown in FIG. 7A. The support system 700 comprises a first bounding layer 702 coupled to a support 704 via a coupling element 705, a second bounding layer 703 onto which the support 704 is coupled, an opening 710, and a first fluid 712. Each of the elements of the support system 700 of FIG. 7B (e.g., the first bounding layer 702, the second bounding layer 703, the support 704, the coupling element 705, the opening 710, the first fluid 712, the first channel 722, etc.) may be of any type described herein.

FIG. 7C illustrates a support system 700 comprising a first bounding layer 702, a support 704 bound to the first bounding layer 702, an opening 710 with a directing element 707 that directs one or more droplets and/or a portion of a solution containing a biological sample into the support 704.

The directing element 707 may comprise a hygroscopic material such as any described herein (e.g., sugar, etc.). The direct element 707 may comprise one or more fibers (such as cellulose fibers) to cause a droplet and/or a portion of the solution containing the biological sample to be directed to the support 704. The direct element 707 may comprise a structure comprising surface features (e.g., steps) that cause a droplet and/or a portion of the solution containing the biological sample to be directed to the support 704.

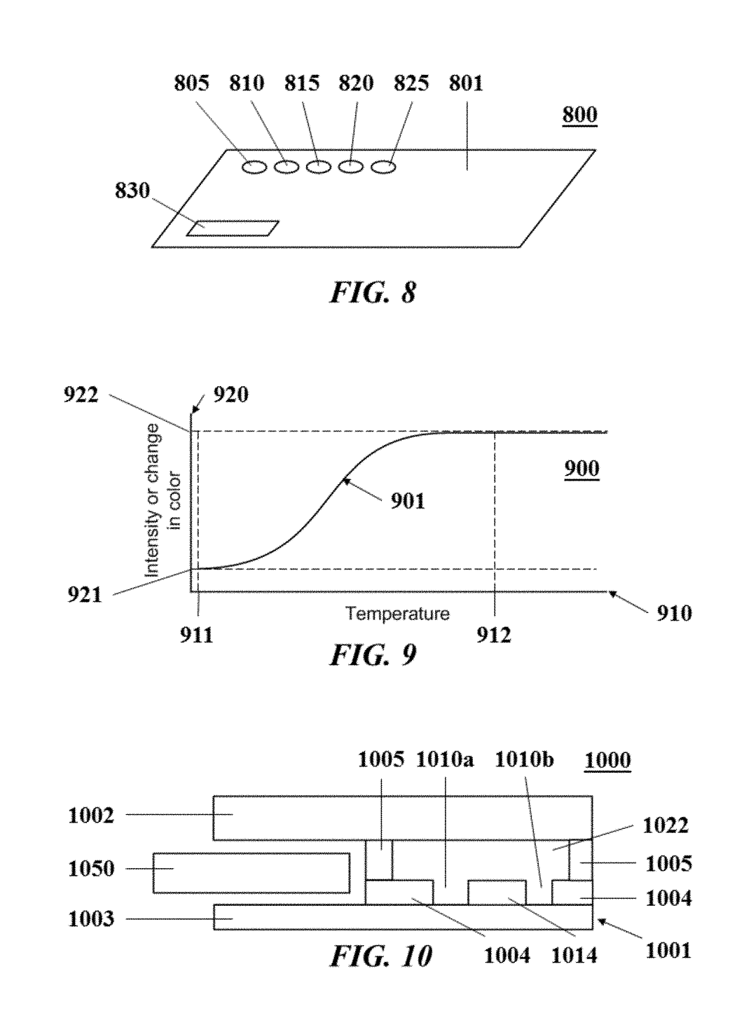

FIG. 8 shows a temperature monitoring system 800 comprising a plurality of temperature indicators 805, 810, 815, 820, 825, 830 coupled to a substrate 801. The substrate 801 may comprise a vessel as described herein, a support as described herein, or the substrate 801 may comprise any surface of any system described herein (e.g., disposed along a vessel surface, a laminate layer a heating layer, etc.). The temperature indictors 805, 810, 815, 820, 825, 830 may individually or collectively comprise one or more resistors, one or more thermocouples, one or more thermistors, one or more diodes, one or more transistors, one or more infrared emitters, one or more detectable moieties (e.g., the temperature indicators 805, 810, 815, 820, 825, 830 may comprise a fluorescent dye or a fluorescent detector), one or more liquid crystals (e.g., one or more thermochromic liquid crystal particles), or one or more temperature sensitive coatings (e.g., a paint, a membrane, a thin film, a layer, etc.). The temperature indicators 805, 810, 815, 820, 825, 830 may individually or collective transmit one or more temperature sensitive parameters. Temperature sensitive parameters may include but are not limited to an electrical resistance, an electrical potential, an electrical current, an open circuit voltage, a color, a light intensity, or any combination thereof. For instance, the temperature indicators 805, 810, 815, 820, 825, 830 may comprise a thermal liquid crystal that reflects light of a first color and/or intensity at a first temperature and light of a second color and/or intensity at a second temperature. The temperature indicators 805, 810, 815, 820, 825, 830 may take on any shape or configuration, such as a circle (as temperature indicators 805, 810, 815, 820, 825 as illustrated take on), an oval an ellipse, a square, a rectangle (as temperature indicator 830 as illustrated shows), a triangle, a line, a particle, two or more particles, or a point (as may be, for example, in cases in which a thermocouple or thermistor is used), or any combination thereof.

One or more temperature indicators 805, 810, 815, 820, 825, 830 may be used such that at least a first temperature indicator (e.g., 805) has a first temperature range (e.g., from about 30° C. to about 50° C.) and a second temperature indicator (e.g., 810) has a second temperature range (e.g., from about 50° C. to about 70° C.). In some embodiments, the first temperature range and the second temperature range have no operative overlap. For example, the first temperature indicator 805 may operatively indicate temperature from 30° C. to less than 50° C. and the second temperature indicator 810 may operatively indicate temperature from 50° C. to less than 70° C. In some embodiments, the first temperature range and the second temperature range have some operative overlap. For example, the first temperature indicator 805 may operatively indicate temperature from 30° C. to 60° C. and the second temperature indicator 810 may operatively indicate temperature from 50° C. to 70° C., such that a portion of the first temperature range and the second temperature of the range are the same. In those embodiments wherein two or more temperature indicators operatively indicate temperature from overlapping temperature ranges, the detector (not illustrated) detecting the temperature may use the results of a first temperature range to calibrate the results of a second temperature range. In those embodiments wherein two or more temperature indicators operatively indicate temperature from overlapping temperature ranges, the detector (not illustrated) detecting the temperature may average the results indicated by the first temperature indicator and the results indicated by the second temperature indicator.

The temperature indicators may individually or collectively have an operative range from about 0° C. to about 10° C., from about 10° C. to about 20° C., from about 20° C. to about 30° C., from about 30° C. to about 40° C., from about 40° C. to about 50° C., from about 50° C. to about 60° C., from about 60° C. to about 70° C., from about 70° C. to about 80° C., from about 80° C. to about 90° C., from about 90° C. to about 100° C., from about 100° C. to about 110° C., from about 110° C. to about 120° C., from about 120° C. to about 130° C., from about 130° C. to about 140° C., from about 140° C. to about 150° C., from about 150° C. to about 160° C., from about 160° C. to about 170° C., from about 170° C. to about 180° C., from about 180° C. to about 190° C., from about 190° C. to about 200° C., from about 200° C. to about 210° C., or the temperature indicators may individually or collectively have an operative range between any two aforementioned values. In some embodiments the temperature indicators may individually or collectively have an operative range from about 0° C. to about 5° C., from about 5° C. to about 10° C., from about 10° C. to about 15° C., from about 15° C. to about 20° C., from about 20° C. to about 25° C., from about 25° C. to about 30° C., from about 30° C. to about 35° C., from about 35° C. to about 40° C., from about 40° C. to about 45° C., from about 45° C. to about 50° C., from about 50° C. to about 55° C., from about 55° C. to about 60° C., from about 60° C. to about 65° C., from about 65° C. to about 70° C., from about 70° C. to about 75° C., from about 75° C. to about 80° C., from about 80° C. to about 85° C., from about 85° C. to about 90° C., from about 90° C. to about 95° C., from about 95° C. to about 100° C., from about 100° C. to about 105° C., from about 105° C. to about 110° C., from about 110° C. to about 115° C., from about 115° C. to about 120° C., from about 120° C. to about 125° C., from about 125° C. to about 130° C., from about 130° C. to about 135° C., from about 135° C. to about 140° C., from about 140° C. to about 145° C., from about 145° C. to about 150° C., from about 150° C. to about 155° C., from about 155° C. to about 160° C., from about 160° C. to about 165° C., from about 165° C. to about 170° C., from about 170° C. to about 175° C., from about 175° C. to about 180° C., from about 180° C. to about 185° C., from about 185° C. to about 190° C., from about 190° C. to about 195° C., from about 195° C. to about 200° C., from about 200° C. to about 205° C.

The temperature indicators 805, 810, 815, 820, 825, 830 may be monitored via any detector described herein. For example, in some embodiments wherein one or more temperature indicators 805, 810, 815, 820, 825, 830 individually or collectively comprises one or more thermal liquid crystal, the temperature indicators 805, 810, 815, 820, 825, 830 may be monitored by a camera.

Though illustrated as coupled to a substrate 801, one or more temperature indicators may be disposed within the sample, the vessel, or one or more droplets of any embodiment. For example, one or more monitoring droplets may individually or collectively comprise one or more of the temperature indicators described herein.

FIG. 9 shows a graph 900 demonstrating an exemplary embodiment of the signal transmitted by a temperature indicator (of any sort described herein) as a function of temperature. The graph 900 comprises two axes, a first axis 910 representing temperature indicated by the temperature indicator (e.g., the temperature of the temperature indicator, the temperature of the substrate to which the temperature indicator is coupled, the temperature of one or more droplets, etc.), and a second axis 920 representing signal intensity (for the illustrated embodiment of this functional temperature indicator, i.e., a thermal liquid crystal, this axis may represent the intensity light, the color, or the change in color, or any combination thereof). The function 901 of the temperature indicator may take on any shape, such as the sigmoidal curve shown in the illustration. The function 901 may have lower 911 and upper 912 operative bounds with respect to temperature such that the signal indicated by the temperature indicator at or below a first temperature 911 has a first response 921 and the signal indicated by the temperature at or above a second temperature 912 has a second response 922. Between the first temperature 911 and the second temperature 912 lies the operative range of the temperature indicator and thus the operative range of responses may lie between the first response 921 of the temperature indicator and the second response 922 of the temperature indicator. The operative temperature range may be of any described herein.

FIG. 10 shows a cross-sectional view of an exemplary embodiment of a temperature monitor 1000 comprising a support system 1001 (similar to those illustrated in FIGS. 7A-7C). The support system may be of any sort described herein. In the illustrated embodiment of FIG. 10 the support system comprises a first bounding layer 1002 and a second bounding layer 1003 coupled via a coupling element 1005 to a support 1004 comprising two wells 1010a and 1010b separated by an intermediate support 1014. A first channel 1022 may demarcated by at least the bounds of the first bounding layer 1002 and the second bounding layer 1003. The temperature of the temperature monitor may be indicated by the temperature indicator 1050 coupled to the temperature monitor. The temperature indicator may be of any type described herein, such as, for example, a thermocouple or a thermistor (e.g., a negative thermal coefficient thermistor, a positive thermal coefficient thermistor, etc.).

In some embodiments, one or more steps of providing materials to a reaction vessel, amplification of nucleic acids, detection of amplified product, and outputting information may be automated by the amplification module. In some embodiments, automation may comprise the use of one or more fluid handlers and associated software. Several commercially available fluid handling systems can be utilized to run the automation of such processes. Non-limiting examples of such fluid handlers include fluid handlers from Perkin-Elmer, Caliper Life Sciences, Tecan, Eppendorf, Apricot Design, and Velocity 11.

In some embodiments, a system or a method to process a biological sample may include a real-time detection instrument. Non-limiting examples of such instruments include a real-time PCR thermocycler, ABI PRISM® 7000 Sequence Detection System, ABI PRISM® 7700 Sequence Detection System, Applied Biosystems 7300 Real-Time PCR System, Applied Biosystems 7500 Real-Time PCR System, Applied Biosystems 7900 HT Fast Real-Time PCR System (all from Applied Biosystems); LightCycler™ System (Roche Diagnostics GmbH); M×3000P™ Real-Time PCR System, M×3005P™ Real-Time PCR System, and M×4000® Multiplex Quantitative PCR System (Stratagene, La Jolla, Calif.); and Smart Cycler System (Cepheid, distributed by Fisher Scientific). In some embodiments, an amplification module may comprise another automated instrument such as, for example, a COBAS® AmpliPrep/COBAS® TaqMan® system (Roche Molecular Systems), a TIGRIS DTS system (Hologic Gen-Probe, San Diego, Calif.), a PANTHER system (Hologic Gen-Probe, San Diego, Calif.), a BD MAX™ system (Becton Dickinson), a GeneXpert System (Cepheid), a Filmarray® (BioFire Diagnostics) system, an iCubate system, an IDBox system (Luminex), an EncompassMDx™ (Rheonix) system, a Liat™ Aanlyzer (IQuum) system, a Biocartis' Molecular Diagnostic Platform system, an Enigma® ML system (Enigma Diagnostics), a T2Dx® system (T2Biosystems), a Verigene® system (NanoSphere), a Great Basin's Diagnostic System, a Unyvero™ System (Curetis), a PanNAT system (Micronics), or a Spartan™ RX system (Spartan Bioscience).

In various aspects, the system may comprise an output module operatively connected to the amplification module. In some embodiments the output module may comprise a device with a processor as described herein. The output module may include input devices as described herein and/or may comprise input electronics for communication with the amplification module. In some embodiments, the output module may be an electronic display, in some cases the electronic display comprising a user interface. In some embodiments, the output module is a communication interface operatively coupled to a computer network such as, for example, the internet. In some embodiments, the output module may transmit information to a recipient at a local or remote location using any suitable communication medium, including a computer network, a wireless network, a local intranet, or the internet. In some embodiments, the output module is capable of analyzing data received from the amplification module. In some cases, the output module includes a report generator capable of generating a report and transmitting the report to a recipient, wherein the report contains any information regarding the amount and/or presence of amplified product as described elsewhere herein. In some embodiments, the output module may transmit information automatically in response to information received from the amplification module, such as in the form of raw data or data analysis performed by software included in the amplification module. Alternatively, the output module may transmit information after receiving instructions from a user. Information transmitted by the output module may be viewed electronically or printed from a printer.

One or more of the input module, biological sample treatment module, amplification module, and output module may be contained within the same device or may comprise one or more of the same components. For example, an amplification module may also comprise an input module, a biological sample treatment module, an output module, or two or more of them.

In other examples, a device comprising a processor may be included in both the input module and the output module. A user may use the device to request that a target nucleic acid be amplified and may also be used to transmit information regarding amplified product to a recipient. In some cases, a device comprising a processor may be included in all four modules, such that the device comprising a processor may also be used to control, provide instructions to, and receive information back from instrumentation (e.g., a thermocycler, a detector, an incubator, a fluid handling device) included in the amplification module or any other module.

Computer control systems

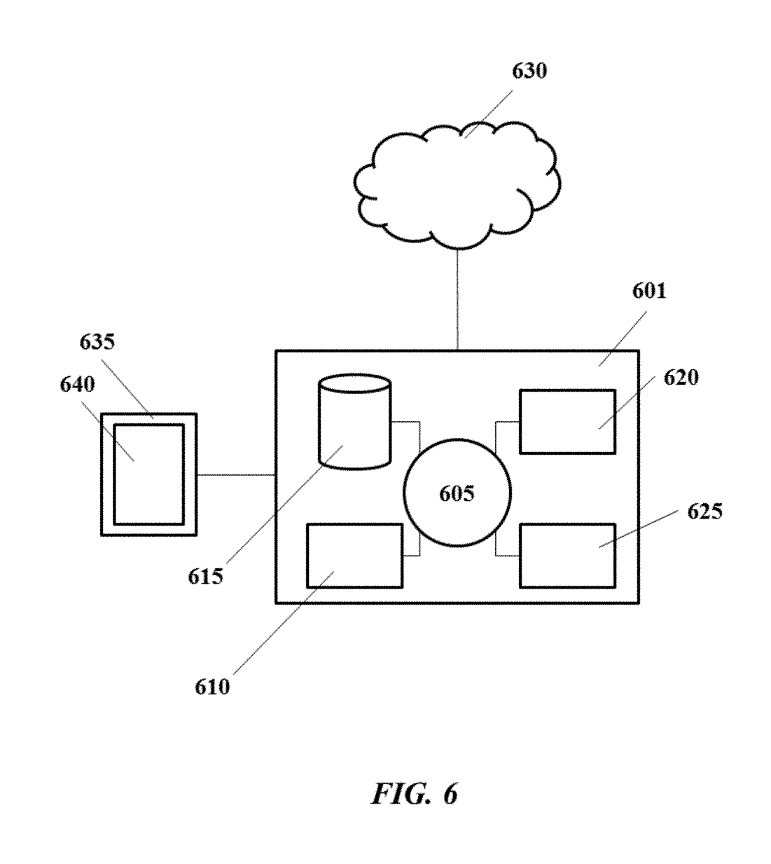

The present disclosure provides computer control systems that are programmed to implement methods of the disclosure. FIG. 6 shows a computer system 601 that is programmed or otherwise configured to process a biological sample (e.g., inductively heat a biological sample) and/or actuate and/or operate components of a system for biological processing, including a system for inductive heating. The computer system 601 can regulate various aspects of processing a biological sample, including inductive heating of a biological sample of the present disclosure, such as, for example, controlling system rotation speeds and number of rotations, controlling electrical current provided to an electromagnet, controlling detector operation, controlling circuitry that is in electrical communication with a device component, monitoring any chemical or biological reaction progress, etc. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, information associated with processing of a biological sample, including information associated with inductive heating (e.g., one or more temperature profiles, system rotation characteristics, magnetic field information, electrical current information) and downstream analysis (e.g., detection data, processed detection data, information about a chemical or biological reaction, etc.). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, control system rotation speeds and number of rotations, control electrical current provided to an electromagnet, control detector operation, control circuitry that is in electrical communication with a device component, monitor chemical and/or biological reaction progress, monitor temperature of a biological sample, etc.

Methods and systems of the present disclosure may be combined with or modified by other methods and systems, such as those described in PCT/CN2014/094914 and PCT/CN2015/095763, each of which is entirely incorporated herein by reference.

### EXAMPLES

Example 1: Inductive Heating

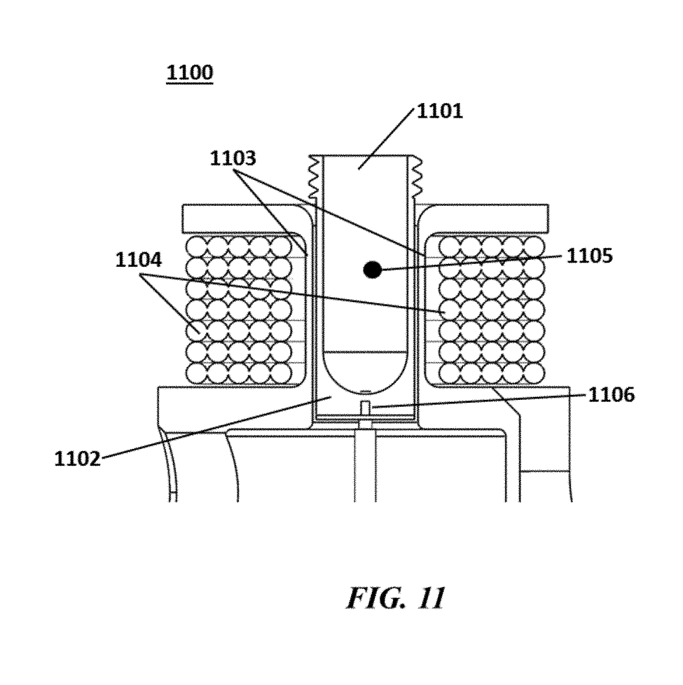

An example inductive heating device 1100 was used to heat a water sample. A cross-sectional view of the device 700 is schematically depicted in FIG. 11. The device included an aluminum tube 1101 situated in a cavity 1102. Cavity 1102 includes a wall 1103, adjacent to which was a coil system 1104 circumscribing the wall 1103. The device 1100 also included thermocouples 1105 and 1106. Thermocouple 1105 was positioned in aluminum tube 1101 near the center of its volume, whereas thermocouple 1106 was positioned below aluminum tube 1101.

During the experiment, 200 microliters (μL) of water was dispensed into tube 1101, positioned within cavity 1102, and sealed with the addition of 150 μL to the top of the water. The water was sealed with the mineral oil in order to prevent evaporation of the water during heating. An electrical current was then pulsed for several cycles through the coil system 1104, thereby generating a pulsed magnetic field through the cavity 1102 and aluminum tube 1101. The magnetic field induced electrical current in the aluminum tube 1101, which generated energy that heated the water sample in aluminum tube 1101. In between magnetic pulses, a source of compressed air (not shown in FIG. 11) was used to cool the water sample. During thermal cycling of the water sample, thermocouples 1105 and 1106 monitored temperatures at their respective locations.

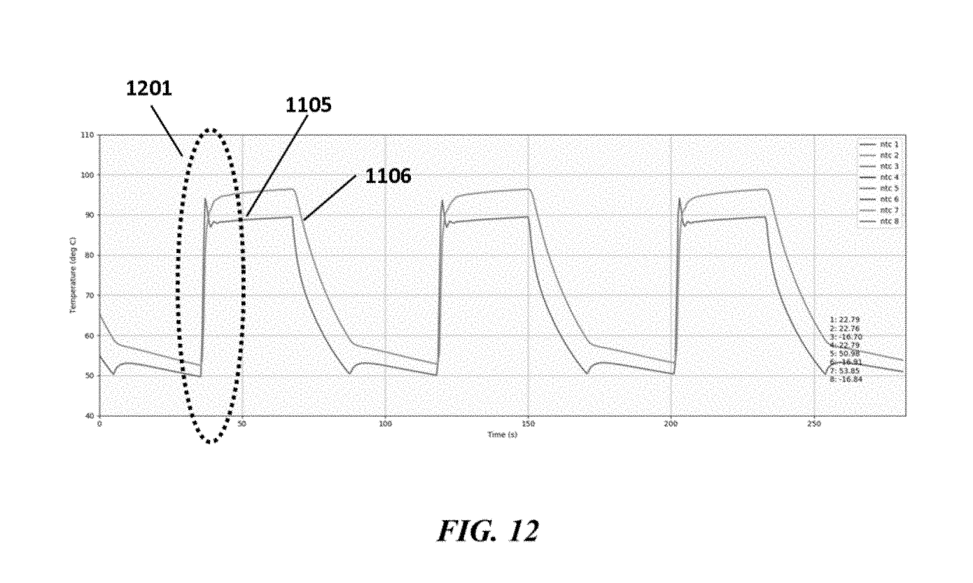

The readouts from thermocouples 1105 and 1106 are graphically depicted in FIG. 12 as temperature vs. time plots. As shown in FIG. 12, temperatures at both thermocouples quickly rose to approximately 90-95° C. and more gradually dropped to baseline values around 50-55° C. after cooling. Heating ramp rates were fast (a representative heating phase identified as 1201 in FIG. 12), with an observed heating ramp rate of approximately 23° C./second (s).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-129. (canceled)

130. A method for processing a biological sample comprising:
(a) providing a solution comprising said biological sample and one or more heating elements in a vessel, wherein said one or more heating elements are dispersed in said solution, and wherein said one or more heating elements generate heat upon inductive coupling to a magnetic field;
(b) bringing said solution in contact with said magnetic field; and
subjecting said solution comprising said biological sample to heating upon inductive coupling of said one or more heating elements to said magnetic field.

131. The method of claim 130, wherein at least a subset of said heating elements is floating, suspended, or dissolved in said solution.

132. The method of claim 130, wherein said solution comprises one or more droplets, and wherein at least a subset of said one or more heating elements is in a given one of said one or more droplets.

133. The method of claim 132, wherein said one or more droplets comprise aqueous droplets in an emulsion.

134. The method of claim 130, wherein said one or more heating elements comprise particles.

135. The method of claim 130, wherein each of said one or more heating elements comprises a polymer and at least one magnetically-active material supported by said polymer, which at least one magnetically-active material generates heat upon inductive coupling to said magnetic field.

136. The method of claim 130, wherein at least one additional heating element is used to subject said solution comprising said biological sample to heating.

137. The method of claim 136, wherein said at least one additional heating element comprises a thermoelectric material.

138. The method of claim 137, wherein said thermoelectric material is optical transparent and electrically conductive.

139. The method of claim 130, further comprising (d) subjecting said solution to cooling.

140. The method of claim 139, further comprising repeating (b)-(d), thereby thermal cycling said solution.

141. The method of claim 140, further comprising conducting a nucleic acid amplification reaction(s) in said solution with the aid of said thermal cycling.

142. A system for processing a biological sample comprising:
a vessel that holds a solution comprising said biological sample and one or more heating elements, wherein said one or more heating elements generate heat upon inductive coupling to a magnetic field;
a magnetic field application unit that provides a magnetic field to said solution; and
a controller operatively coupled to said magnetic field application unit, wherein said controller is programmed to direct said magnetic field application unit to apply said magnetic field to said solution, thereby subjecting said solution to heating upon inductive coupling of said one or more heating elements to said magnetic field.

143. The system of claim 142, wherein at least a subset of said one or more heating elements is floating, suspended, or dissolved in said solution.

144. The system of claim 142, wherein said vessel is selected from the group consisting of a tube, a cuvette, a chamber, a beaker, a reservoir, a flow channel, a capillary tube, a well, a multi-well plate, a bottle and a flask.

145. The system of claim 142, wherein said solution comprises one or more droplets, and wherein at least a subset of said one or more heating elements is in a given one of said one or more droplets.

146. The system of claim 142, wherein said magnetic field application unit comprises a permanent magnet or an electromagnet.

147. The system of claim 142, further comprising a cooling unit that subjects said solution to cooling.

148. The system of claim 142, wherein said controller comprises one or more computer processors that are individually or collectively programmed to alternately and sequentially position said solution in said magnetic field and in a cooling zone.

149. The system of claim 142, wherein said vessel is rotatable with respect to said magnetic field application unit, or vice versa.
